(19) United States
(12) Patent Application Publication
(10) Pub. No.: US 2019/0298310 A1
(43) Pub. Date: Oct. 3, 2019

(54) ULTRASONIC DIAGNOSTIC APPARATUS AND METHOD OF CONTROLLING THE SAME

(71) Applicant: Konica Minolta, Inc., Tokyo (JP)

(72) Inventor: MASARU FUSE, Osaka (JP)

(21) Appl. No.: 16/251,962

(22) Filed: Jan. 18, 2019

(30) Foreign Application Priority Data
Feb. 6, 2018 (JP) 2018-018957

(51) Int. Cl. A61B 8/08; G01S 7/52

(52) U.S. Cl. CPC A61B 8/485; G01S 7/52022; A61B 8/5276; A61B 8/085; G01S 7/52042

(57) ABSTRACT

An ultrasonic diagnostic apparatus to which a probe including a plurality of oscillators is connectable and that causes the probe to transmit a push wave including an ultrasonic beam to focus into an object to be examined, to detect a propagation speed of a shear wave, includes: a push-wave pulse transmitter that supplies a push-wave pulse to each of a plurality of transmission oscillators, to cause the plurality of transmission oscillators to sequentially transmit a plurality of push waves to focus onto a plurality of transmission focuses, a detection-wave pulse transmitter that supplies a detection-wave pulse to part or all of the plurality of oscillators, to cause the plurality of oscillators to transmit a detection wave to pass through a region of interest; and a propagation information analyzer that calculates propagation-speed frame data of a shear wave in the region of interest.

The entire disclosure of Japanese patent Application No. 2018-018957, filed on Feb. 6, 2018, is incorporated herein by reference in its entirety.

### BACKGROUND

### Technological Field

The present disclosure relates to an ultrasonic diagnostic apparatus and a method of controlling the ultrasonic diagnostic apparatus, and particularly relates to, with a shear wave, analysis of the propagation speed of the shear wave in tissue and measurement of the elastic modulus of the tissue.

### DESCRIPTION OF THE RELATED ART

An ultrasonic diagnostic apparatus is a medical examination apparatus that transmits an ultrasonic wave from a plurality of oscillators included in an ultrasonic probe to the inside of an object to be examined, receives an ultrasonic reflected wave (echo) caused due to a difference in acoustic impedance of the tissue of the object to be examined, generates an ultrasonic tomographic image indicating the structure of the internal tissue of the object to be examined, on the basis of an acquired electric signal, and displays the ultrasonic tomographic image.

In recent years, tissue elastic-modulus measurement with application of such ultrasonic diagnostic technique, has been widely used for examination (shear wave speed measurement (SWSM), hereinafter, referred to as “ultrasonic elastic-modulus measurement”). Because the hardness of a tumor discovered in an organ or body tissue can be measured noninvasively and easily, the ultrasonic elastic-modulus measurement can be usefully used for inspection of the hardness of a tumor in screening examination for cancer or evaluation for hepatic fibrosis in examination for liver disease.

In the ultrasonic elastic-modulus measurement, an region of interest (ROI) in an object to be examined is determined, and a push wave including an ultrasonic wave to focus onto a specific part in the object to be examined is transmitted from a plurality of oscillators (focused ultrasonic wave or acoustic radiation force impulse (ARFI)). After that, transmission of an ultrasonic wave for detection (hereinafter, referred to as a “detection wave”) and reception of a reflected wave are repeated a plurality of times. Then, propagation analysis of a shear wave caused by the acoustic radiation pressure of the push wave, is performed to calculate the propagation speed of the shear wave expressing the elastic modulus of tissue. Then, for example, the distribution of tissue elasticity is rendered in imaging, so that an elasticity image can be displayed (e.g., JP 2006-500089 A).

For examination with the ultrasonic elastic-modulus measurement, it is desired that rendering an elasticity image in high quality with an increase in the S/N of a signal for acquiring the elasticity image, facilitates verification of a delicate lesion.

The wave-front shape of a shear wave to be used in the ultrasonic elastic-modulus measurement, has a substantially spherical-wave shape around a transmission focus. In order to acquire the propagation speed of the shear wave accurately, it is necessary to observe displacement on a line orthogonal to the propagation direction of the shear wave at a depth identical to the depth of the transmission focus of the push wave. Thus, the region in which the value of elasticity can be measured with one push wave, is narrow.

Therefore, it has been suggested that transmission of a plurality of push waves to different positions in an object to be examined generates a shear wave having a wide wave front in a region of interest, artificially, to expand an elasticity measurement region (e.g., JP 2016-7315 A and JP 2016-22249 A).

However, it is necessary to generate an intense push wave for generation of a shear wave in the ultrasonic elastic-modulus measurement. Thus, a bulk-power source circuit is required, and furthermore a larger-scale source circuit is required in order to generate a plurality of push waves. Therefore, power saving is required together with improvement of the accuracy of propagation analysis and expansion of a measurement region.

### SUMMARY

The present disclosure has been made in consideration of the problem, and an object of the present disclosure is to improve the reliability of measurement in a region of interest with less power consumption in ultrasonic elastic-modulus measurement.

To achieve the abovementioned object, according to an aspect of the present invention, there is provided an ultrasonic diagnostic apparatus to which a probe including a plurality of oscillators arranged linearly is connectable and that causes the probe to transmit a push wave including an ultrasonic beam to focus into an object to be examined, to detect a propagation speed of a shear wave generated by an acoustic radiation pressure of the push wave, and the ultrasonic diagnostic apparatus reflecting one aspect of the present invention comprises: a push-wave pulse transmitter that supplies, a plurality of times, a push-wave pulse that is set with a predetermined phase and has a predetermined time length, to each of a plurality of transmission oscillators selected from the plurality of oscillators, to cause the plurality of transmission oscillators to sequentially transmit a plurality of push waves to focus onto a plurality of transmission focuses different in position in a depth direction of the object to be examined; a detection-wave pulse transmitter that supplies, after the transmission of the plurality of push waves, a detection-wave pulse to part or all of the plurality of oscillators, to cause the plurality of oscillators to transmit, a plurality of times, a detection wave to pass through a region of interest expressing a range to be analyzed in the object to be examined; and a propagation information analyzer that calculates propagation-speed frame data of a shear wave in the region of interest, based on a reflected detection wave received on a time series basis by the plurality of oscillators, the reflected wave corresponding to each of the plurality of detection waves, wherein the push-wave pulse transmitter causes the transmission of the plurality of push waves such that an interval between the transmission focuses adjacent to each other is larger in a deeper portion of the object to be examined and is smaller in a shallower portion of the object to be examined and a ratio of a depth of each transmission focus to an array length of the plurality of transmission oscillators is larger in the deeper portion of the object to be examined and is smaller in the shallower portion of the object to be examined.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a schematic view of the overview of an SWS sequence with an ultrasonic elastic-modulus measurement method in an ultrasonic diagnostic apparatus according to an embodiment;

FIG. 2 is a functional block diagram of an ultrasonic diagnostic system including the ultrasonic diagnostic apparatus:

FIG. 3A is a schematic view of the position of the transmission focus of a push wave to be generated by a push-wave pulse generator;

FIG. 3B is a schematic view of the configuration overview of a detection-wave pulse to be generated by a detection-wave pulse generator;

FIG. 4A is a functional block diagram of the configuration of a transmitter;

FIG. 4B is a functional block diagram of the configuration of a detection-wave receiver:

FIGS. 5A to 5C are schematic charts of the application timing of a push-wave pulse:

FIGS. 6A to 6D are schematic views of the overview of the push wave;

FIG. 7A is a schematic view of the overview of detection-wave transmission;

FIG. 7B is a schematic view of the overview of reflected detection-wave reception;

FIG. 8 is a schematic view of the overview of a method of calculating the propagation path of an ultrasonic wave, in a delay processor.

FIG. 9 is a functional block diagram of the configurations of a displacement detector, a propagation information analyzer, and an elastic-modulus calculator;

FIG. 10 is a schematic view of the overview of an integrated SWS sequence process in the ultrasonic diagnostic apparatus;

FIG. 11 is a flowchart of the operation of ultrasonic elastic-modulus calculation in the ultrasonic diagnostic apparatus;

FIGS. 12A to 12E are schematic views of the transition of generation of a shear wave due to a push wave:

FIG. 13 is a schematic view of the operation of displacement detection and propagation analysis of the shear wave;

FIG. 14 is a flowchart of the operation of propagation information analysis of the shear wave in the ultrasonic diagnostic apparatus;

FIGS. 15A to 15F are schematic charts of the operation of the propagation analysis of the shear wave;

FIG. 16 is a flowchart of the operation of elastic-modulus calculation in the ultrasonic diagnostic apparatus;

FIG. 17 is a flowchart of the operation of reception beam forming;

FIG. 18 is a flowchart of the operation of acoustic-line-signal frame data generation:

FIG. 19 is a flowchart of the operation of acoustic-line-signal data generation for an observation point;

FIG. 20A is a simulated result of an aspect of the shear wave due to the push wave in the ultrasonic diagnostic apparatus for a shallower portion of the object to be examined;

FIG. 20B is a simulated result of an aspect of the shear wave due to the push wave in the ultrasonic diagnostic apparatus for a deeper portion of the object to be examined;

FIGS. 21A and 21B are each a schematic view of an aspect of propagation of the shear wave based on the push wave in the ultrasonic diagnostic apparatus;

FIGS. 22A and 22B are each a schematic view of a measurable region with the shear wave based on the push wave in the ultrasonic diagnostic apparatus;

FIGS. 23A to 23D are schematic views of aspects of the measurable region with the shear wave based on the push wave, and FIGS. 23A, 23B, 23C, and 23D illustrate Example. Comparative Example 1, Comparative Example 2, and Modification, respectively;

FIG. 24 is a functional block diagram of the configurations of a displacement detector, a propagation information analyzer, and an elastic-modulus calculator in an ultrasonic diagnostic apparatus according to a second embodiment;

FIGS. 25A and 25B are a schematic view and a graph each illustrating the operation of propagation analysis of a shear wave in the ultrasonic diagnostic apparatus;

FIG. 26 is a flowchart of the operation of propagation information analysis of the shear wave in the ultrasonic diagnostic apparatus; and

FIG. 27 is a flowchart of the operations at steps S1532A and S1533A in FIGS. 25A and 25B.

### DETAILED DESCRIPTION OF EMBODIMENTS

Hereinafter, one or more embodiments of the present invention will be described with reference to the drawings. However, the scope of the invention is not limited to the disclosed embodiments.

### Details of Embodiments of Invention

In ultrasonic elastic-modulus measurement with an elasticity measurement technique, because the region in which the value of elasticity can be measured with a push wave, is narrow, it is considered that transmission of a plurality of push waves to different positions in an object to be examined generates a shear wave having a wide wave front in a region of interest, artificially, to improve the accuracy of propagation analysis.

In order to ensure a wide measurement region, for example, a Mach Cone method can be used, in which a plurality of shear-wave sources is substantially simultaneously generated with successive incidence of a plurality of push waves, and respective spherical waves generated from the wave sources are combined to create a virtual plane wave. Typically, the Mach Cone method has the respective positions in the depth direction of transmission focuses of the push waves set at regular intervals and the transmission intervals of the push waves set identically. The method enables performance of elasticity measurement in a wider range than that with one push wave. In a case where the Mach Cone method is used, it is not necessary to perform integrated SWS sequence processing of performing ultrasonic elastic-modulus measurement (SWS sequence) with one push wave a plurality of times to integrate respective results acquired from the SWS sequences.

Meanwhile, in the ultrasonic elastic-modulus measurement with the elasticity measurement technique, it is necessary to generate an intense push wave for generation of a shear wave. Thus, a bulk-power source circuit is required, and power saving is required together with improvement of the accuracy of propagation analysis.

Furthermore, because required power in the Mach Cone method doubles by the number of push waves, extremely large-capacity power source is necessary for practical use. Thus, there is a restriction that only large machines can be equipped with the ultrasonic elastic-modulus measurement.

Therefore, the inventors have zealously considered that the same function can be implemented on smaller machines with reduction of the power necessary for the ultrasonic elastic-modulus measurement or that, due to optimization of power distribution, high-accuracy measurement is achieved by improvement of the efficiency of detecting a shear wave with power consumption at a similar level.

In general, for example, in B mode, in order to ensure the uniformity in image quality, the transmission numerical aperture of BF is typically changed in accordance with a focal depth such that a so-called “F number” is constant.

In contrast to this, the inventors have considered that, favorably, the numerical aperture is ensured as large as possible regardless of depth because transmission of a push wave requires causing large physical displacement. In this case, a substantially ideal point wave source can be formed at a shallow portion of an object to be examined, but a wave source expanding in the depth direction is formed at a deep portion of the object to be examined. Therefore, the shear wave has a spherical-wave shape at the shallow portion, but has a shape close to a plane wave having a gentle curvature, at the deep portion. The inventors have focused on and considered the difference in the wave-front shape of the shear wave depending on the depth of the transmission focus of the push wave. As a result, the inventors have conceived that narrowing the intervals in focus at the shallow portion and broadening the intervals in focus at the deep portion, allow proper coupling of elasticity measurable regions differing depending on the transmission focus, so that wide-range measurement can be performed as a whole. For achievement of the configuration, the inventors have conceived that controlling narrowing of the transmission interval between push waves for transmission to the shallow portion and broadening of the transmission interval for transmission to the deep portion, enables reduction of the total number of push waves and reduction of the power necessary for the ultrasonic elastic-modulus measurement, resulting in an ultrasonic diagnostic apparatus and a method of controlling the ultrasonic diagnostic apparatus according to an embodiment.

### First Embodiment

<Overview of Ultrasonic Elastic-Modulus Measurement>

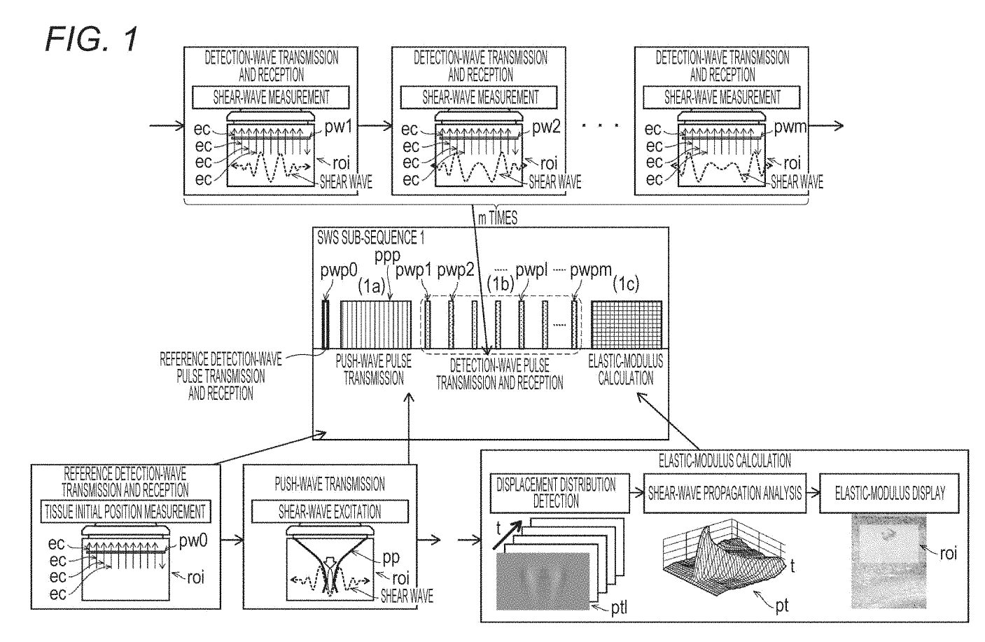

An ultrasonic diagnostic apparatus 100 performs processing of calculating the propagation speed of a shear wave expressing the elastic modulus of tissue, with an ultrasonic elastic-modulus measurement method. FIG. 1 is a schematic view of the overview of an SWS sequence with the ultrasonic elastic-modulus measurement method in the ultrasonic diagnostic apparatus 100. As indicated in the central box of FIG. 1, the processing of the ultrasonic diagnostic apparatus 100 includes processes of “reference detection-wave pulse transmission and reception”, “push-wave pulse transmission”, “detection-wave pulse transmission and reception”, and “elastic-modulus calculation”.

The process of “reference detection-wave pulse transmission and reception” includes: transmitting a reference detection-wave pulse pwp0 to an ultrasonic probe; causing a plurality of oscillators to transmit a detection wave pw0 to and to receive a reflected wave ec from a range corresponding to a region of interest roi in an object to be examined; and generating an acoustic line signal serving as a reference at the initial position of tissue.

The process of “push-wave pulse transmission” includes: transmitting a push-wave pulse ppp to the ultrasonic probe; causing the plurality of oscillators to transmit a push wave pp including an ultrasonic wave to focus onto a specific part in the object to be examined; and exciting a shear wave in the tissue of the object to be examined.

The process of “detection-wave pulse transmission and reception” includes: transmitting a detection-wave pulse pwpl to the ultrasonic probe (l is a natural number of 1 to m that is the number of times a detection-wave pulse pwp is transmitted, but the detection-wave pulse pwpl is provided in a case where the numbers are not distinguished); causing the plurality of oscillators to transmit a detection wave pwl and to receive a reflected wave ec a plurality of times; and measuring the shear wave. The process of “elastic-modulus calculation” includes: first calculating, on a time series basis, the displacement distribution ptl of the tissue accompanied with the propagation of the shear wave caused by the acoustic radiation pressure of the push wave; next performing shear-wave propagation analysis of calculating the propagation speed of the shear wave expressing the elastic-modulus of the tissue from a time-series variation in the acquired displacement distribution ptl; and last rendering the distribution of tissue elasticity, for example, in imaging to display an elasticity image (elastography).

A series of processes accompanied with shear-wave excitation for one time based on the transmission of the push wave pp described above, is defined as an “SWS sequence” (shear wave speed (SWS)), and a process including a plurality of “SWS sequences” integrated, is defined as an “integrated SWS sequence”.

<Ultrasonic Diagnostic System 1000>

1. Configuration Overview

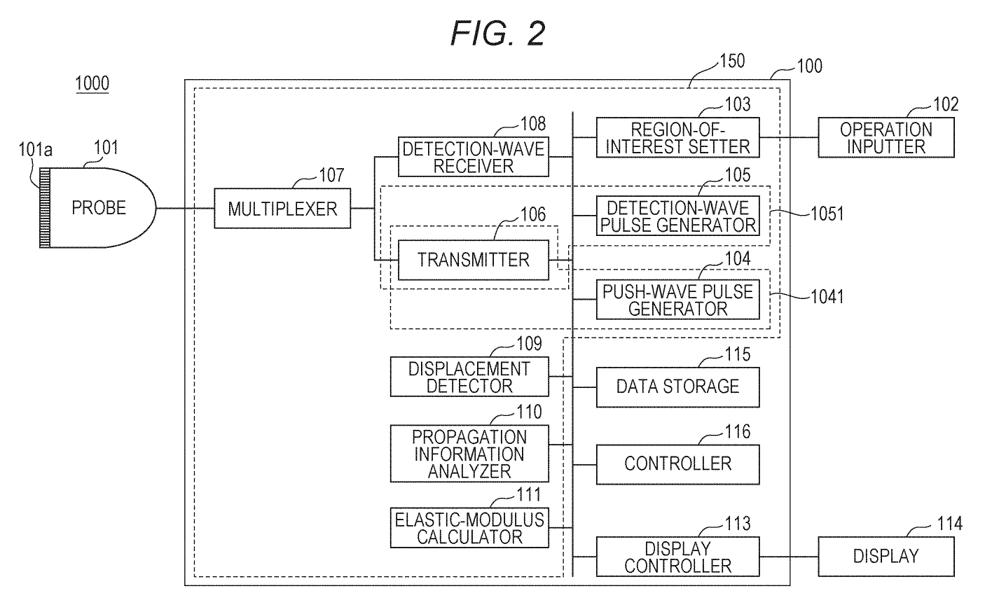

An ultrasonic diagnostic system 1000 including the ultrasonic diagnostic apparatus 100 according to the first embodiment, will be described with reference to the drawings. FIG. 2 is a functional block diagram of the ultrasonic diagnostic system 1000 according to the first embodiment. As illustrated in FIG. 2, the ultrasonic diagnostic system 1000 includes: an ultrasonic probe 101 (hereinafter, referred to as a “probe 101”) including a plurality of oscillators (oscillator array) 101a that is arranged linearly on a leading-end face, transmits an ultrasonic wave to an object to be examined, and receives a reflected wave thereof; the ultrasonic diagnostic apparatus 100 that causes the probe 101 to transmit and receive the ultrasonic wave, to generate an ultrasonic image on the basis of an output signal from the probe 101; an operation inputter 102 that receives an operation input from an examiner; and a display 114 that displays the ultrasonic image on a screen. The probe 101, the operation inputter 102, and the display 114 are each connectable to the ultrasonic diagnostic apparatus 100.

Next, each element connected to the ultrasonic diagnostic apparatus 100 externally, will be described.

2. Probe 101

The probe 101 includes the oscillator array (101a) including the plurality of oscillators 101a arranged, for example, in a one-dimensional direction (hereinafter, referred to as an “oscillator array direction”). The probe 101 converts a pulse-shaped electric signal (hereinafter, referred to as a “transmission signal”) supplied from a transmitter 106 to be described later, into a pulse-shaped ultrasonic wave. With the outer face on the oscillator side of the probe 101, abutting on a skin surface of the object to be examined through, for example, ultrasonic gel, the probe 101 transmits an ultrasonic beam including a plurality of ultrasonic waves emitted from the plurality of oscillators, to an object to be measured. Then, the probe 101 receives a plurality of reflected detection waves (hereinafter, referred to as “reflected waves”) from the object to be examined. The plurality of oscillators 101a converts the reflected waves into respective electric signals. The probe 101 supplies the electric signals to the ultrasonic diagnostic apparatus 100.

3. Operation Inputter 102

The operation inputter 102 receives, from the examiner, various operation inputs for various settings or operations to the ultrasonic diagnostic apparatus 100, and outputs the various operation inputs to a controller 116 of the ultrasonic diagnostic apparatus 100.

For example, the operation inputter 102 may include a touch panel integrally formed with the display 114. In this case, performance of a touch operation or a drag operation to an operation key displayed by the display 114, enables the various settings or operations to the ultrasonic diagnostic apparatus 100. Thus, the ultrasonic diagnostic apparatus 100 is operable through the touch panel. For example, the operation inputter 102 may include a keyboard including keys for the various operations, an operation panel including buttons or levers for the various operations, or a mouse.

4. Display 114

The display 114 including a so-called display device for image display, displays an image output from a display controller 113 to be described later, onto the screen. For example, a liquid crystal display, a CRT, or an organic EL display can be used for the display 114.

<Configuration Overview of Ultrasonic Diagnostic Apparatus 100>

Next, the ultrasonic diagnostic apparatus 100 according to the first embodiment, will be described.

The ultrasonic diagnostic apparatus 100 includes: a multiplexer 107 that selects an oscillator to be used in transmission or reception individually from the plurality of oscillators 101a of the probe 101 and ensures an input and an output for the selected oscillator; the transmitter 106 that controls the timing of applying high voltage to each oscillator 101a of the probe 101 in order to transmit an ultrasonic wave; and a detection-wave receiver 108 that performs reception beam forming on the basis of a reflected wave received by the probe 101, to generate an acoustic line signal.

The ultrasonic diagnostic apparatus 100 includes: a region-of-interest setter 103 that sets the region of interest roi expressing a range to be analyzed in the object to be examined, with respect to the plurality of oscillators 101a, on the basis of an operation input from the operation inputter 102; a push-wave pulse generator 104 that causes transmission of the push-wave pulse ppp to the plurality of oscillators 101a; and a detection-wave pulse generator 105 that causes transmission of the detection-wave pulse pwpl a plurality of times (m), following the push-wave pulse ppp.

The ultrasonic diagnostic apparatus 100 includes: a displacement detector 109 that detects displacement of the tissue in the region of interest roi, from the acoustic line signal: a propagation information analyzer 110 that performs propagation information analysis of the shear wave from the detected displacement of the tissue, calculates the wave-front arrival time of the shear wave to each observation point in the region of interest roi, and calculates the propagation speed of the shear wave; and an elastic-modulus calculator 111 that calculates the elastic modulus at each observation point in the region of interest roi.

The ultrasonic diagnostic apparatus 100 includes: a data storage 115 that stores, for example, the acoustic line signal output by the detection-wave receiver 108, displacement-amount data output by the displacement detector 109, wave-front data, wave-front arrival-time data, and speed-value data output by the propagation information analyzer 110, and elastic-modulus data output by the elastic-modulus calculator 111; a display controller 113 that arranges a display image and causes the display 114 to display the display image; and the controller 116 that controls each constituent element.

From the constituent elements, the multiplexer 107, the transmitter 106, the detection-wave receiver 108, the region-of-interest setter 103, the push-wave pulse generator 104, the detection-wave pulse generator 105, the displacement detector 109, the propagation information analyzer 110, and the elastic-modulus calculator 11l are included in an ultrasonic signal processing circuit 150.

For example, the elements included in the ultrasonic signal processing circuit 150, the controller 116, and the display controller 113 are each achieved by a hardware circuit, such as a field programmable gate array (FPGA) or an application specific integrated circuit (ASIC). Alternatively, the constituent elements each may be achieved by a programmable device, such as a central processing unit (CPU), a general-purpose computing on graphics processing unit (GPGPU), or a processor, and software. The constituent elements can be made as one circuit component, or can be made as an assembly of a plurality of circuit components. One circuit component can be made with a combination of a plurality of constituent elements, or an assembly of a plurality of circuit components can be made.

The data storage 115 including a computer-readable recording medium, can adopt, for example, a flexible disk, a hard disk, an MO, a DVD, a DVD-RAM, or a semiconductor memory. The data storage 115 may include a storage device connected to the ultrasonic diagnostic apparatus 100 externally.

Note that the ultrasonic diagnostic apparatus 100 according to the first embodiment is not limited to having the configuration illustrated in FIG. 2. For example, no multiplexer 107 may be provided, or the probe 101 may include the transmitter 106 and the detection-wave receiver 108 or may have part thereof built in.

<Configuration of Each Part of Ultrasonic Diagnostic Apparatus 100>

Next, the configuration of each block included in the ultrasonic diagnostic apparatus 100, will be described.

1. Region-of-Interest Setter 103

In general, with the display 114 displaying a B-mode image that is a tomographic image of the object to be examined in real time acquired by the probe 101, an operator specifies the range to be analyzed in the object to be examined, with the B-mode image displayed by the display 114 as an indicator, and then inputs the range to be analyzed into the operation inputter 102. The region-of-interest setter 103 sets, as an input, information specified by the operator from the operation inputter 102, and outputs the information to the controller 116. At this time, the region-of-interest setter 103 may set the region of interest roi expressing the range to be analyzed in the object to be examined, with respect to the position of the oscillator array (101a) including the plurality of oscillators 101a in the probe 101. For example, the region of interest roi may be all region or a partial region in a detection-wave irradiation region Ax including the oscillator array (101a) including the plurality of oscillators 101a.

2. Push-Wave Pulse Generator 104

The push-wave pulse generator 104 inputs information indicating the region of interest roi, from the controller 116, and sets a specific point at a predetermined position in the region of interest roi. Then, the push-wave pulse generator 104 causes the transmitter 106 to transmit a push-wave pulse pppn (n=1 to nₘₐₓ) to the plurality of oscillators 101a a plurality of times (nₘₐₓ), to cause the plurality of oscillators 101a to transmit a push wave ppn (n=1 to nₘₐₓ) including an ultrasonic beam to focus onto a specific part in the object to be examined corresponding to the specific point (hereinafter, referred to as a “transmission focus FPn” (n=1 to nₘₐₓ). Thus, shear-wave excitation occurs in the specific part in the object to be examined. At this time, the number of times (nₘₐₓ) the push-wave pulse pppn is transmitted may range, for example, from three to eight, more preferably, from four to six. However, needless to say, nₘₐₓ is not limited to the above, and thus can be appropriately changed.

Specifically, the push-wave pulse generator 104 determines, on the basis of the information indicating the region of interest roi, the position of the transmission focus PPn of the push wave and the oscillator array that transmits the push wave ppn (hereinafter, referred to as a “push-wave transmission oscillator array Pxn”) as indicated below.

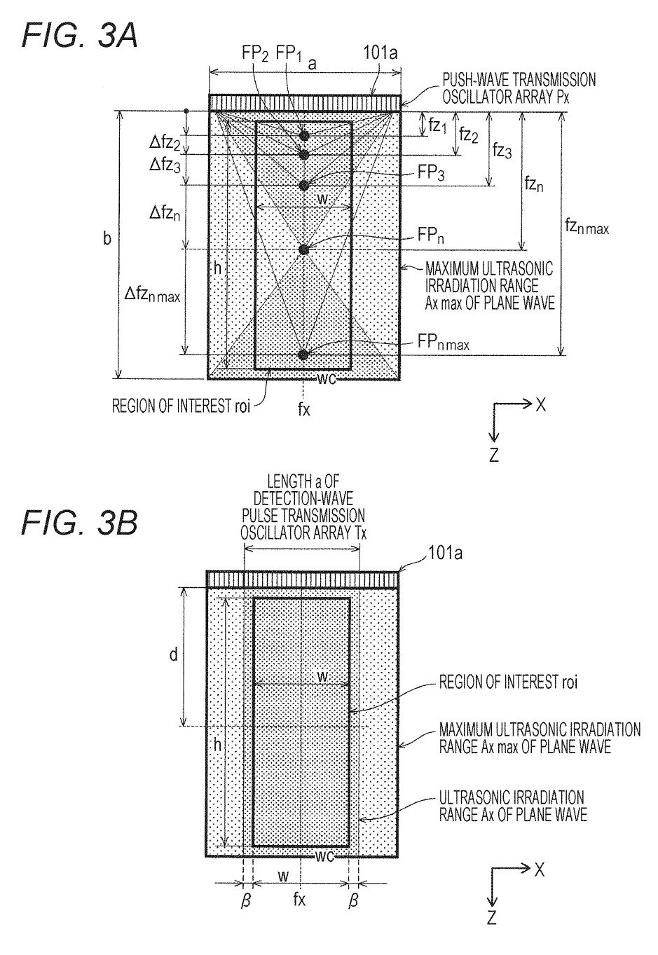

FIG. 3A is a schematic view of the position of the transmission focus FPn of the push wave ppn to be generated by the push-wave pulse generator 104. A case where the length w in the array direction and the length h in the depth direction of the object to be examined of the region of interest roi are not more than the length a in the array direction and the length b in the depth direction of the object to be examined of the ultrasonic radiation range of a plane wave, respectively, and the region of interest roi is set in the vicinity of the center of the ultrasonic radiation range, will be exemplarily described. In the present embodiment, as illustrated in FIG. 3A, for the position of the transmission focus FPn, for example, the array-direction transmission focus position fx agrees with the array-direction central position we of the region of interest roi. The depth-direction transmission focus position fzn (n=1 to nₘₐₓ) has a larger interval Δfzn between the adjacent transmission focuses fan in a deeper portion of the object to be examined, and has a smaller interval Δfzn between the adjacent transmission focuses fzn in a shallower portion of the object to be examined.

The push-wave transmission oscillator array Pt is set on the basis of the depth-direction transmission focus position fan. In the present embodiment, the length of the push-wave pulse transmission oscillator array Pxn (n=1 to nₘₐₓ) is set to the array length a of all the plurality of oscillators 101a. This arrangement allows the ratio of the depth of the transmission focus FPn to the array length of the push-wave transmission oscillator array Px, to be larger in the deeper portion of the object to be examined and to be smaller in the shallower portion of the object to be examined, in accordance with the depth of the transmission focus FPn (coordinate in the depth direction of the depth-direction transmission focus position fzn).

Information indicating the position of the transmission focus FPn and the push-wave transmission oscillator array Pxn, is output as a transmission control signal together with the pulse width PWn and the application start time PTn of the push-wave pulse pppn, to the transmitter 106. The time interval Pin of the application start time PTn may be included. Note that the pulse width PWn, the application start time PTn, and the time interval Pin of the push-wave pulse pppn, will be described later.

Note that, in the configuration, the length of the push-wave pulse transmission oscillator array Pxn is not limited to the above, and thus may be appropriately changed depending on a mode of a part to be examined of the object to be examined as long as the condition in which the ratio of the depth of the transmission focus FPn to the array length of the push-wave transmission oscillator array Px, is larger in the deeper portion of the object to be examined and is smaller in the shallower portion of the object to be examined, is satisfied. For example, the push-wave pulse transmission oscillator array Pxn may be part of the array of the plurality of oscillators 101a. The length of the push-wave pulse transmission oscillator array Pxn may vary depending on the transmission focus FPn.

The positional relationship between the region of interest roi and the transmission focus FPn is not limited to the above, and thus may be appropriately changed depending on a mode of a part to be examined of the object to be examined.

For example, the example illustrated in FIG. 3A may be changed such that, for the position of the transmission focus FP, the array-direction transmission focus position fx is offset from the array-direction central position we of the region of interest roi to the positive or negative of the X axis. In this case, the array-direction center of the region-of-interest width w is different from the array-direction center of the oscillator array. Furthermore, for the position of the transmission focus FP, the array-direction transmission focus position fx may be offset from the array-direction central position we of the region of interest roi to the positive or negative of the X axis, so as to be located outside the region of interest roi.

In a case where the region-of-interest width w is relatively large, a plurality of push waves may be generated in which the array-direction transmission focus position fx of the transmission focus FPn varies depending on the transmission focus FPn.

The transmission focus FP may be set at a predetermined position in proximity to the region of interest roi and outside the region of interest roi. At this time, in a case where the transmission focus FP is set in proximity to the region of interest roi, the transmission focus FP is set at a distance from the region of interest roi at which the shear wave can arrive at the region of interest roi.

Note that the “focusing” of the ultrasonic beam of the push wave indicates a focus beam including the ultrasonic beam focused, namely, that an area to be irradiated with the ultrasonic beam decreases after the transmission and has a minimum value at a specific depth, and thus is not limited to a case where the ultrasonic beam focuses on one point. In this case, the “transmission focus FP” indicates the center of the ultrasonic beam at the depth at which the ultrasonic beam focuses.

In the present specification, in a case where the order of transmission (n) is not distinguished for the push-wave pulse pppn, the push wave ppn, the push-wave transmission oscillator array Pxn, the transmission focus FPn, the depth-direction transmission focus position fan, the interval Δfzn between the adjacent transmission focuses fan, the pulse width PWn and the application start time PTn of the push-wave pulse pppn, and the time interval PIn of the application start time PTn, the n will be omitted.

3. Detection-Wave Pulse Generator 105

The detection-wave pulse generator 105 inputs the information indicating the region of interest roi from the controller 116, and causes the transmitter 106 to transmit the detection-wave pulse pwpl to the plurality of oscillators 101a a plurality of times, to cause the plurality of oscillators 101a belonging to a detection-wave pulse transmission oscillator array Tx, to transmit the detection wave pw such that the ultrasonic beam passes through the region of interest roi. Specifically, on the basis of the information indicating the region of interest roi, the detection-wave pulse generator 105 determines the oscillator array to which the detection-wave pulse pwpl is transmitted such that the ultrasonic beam passes through the region of interest roi (hereinafter, referred to as a “detection-wave transmission oscillator array Tx”). At this time, the number of times (m) the detection-wave pulse pwpl is transmitted may range, for example, from 30 to 100. The interval at which the detection-wave pulse pwpl is transmitted may range, for example, from 100 to 150 μsec. However, needless to say, the application conditions are not limited to the above, and thus can be appropriately changed.

FIG. 3B is a schematic view of the configuration overview of the detection-wave pulse pwpl to be generated by the detection-wave pulse generator 105. As illustrated in FIG. 3B, the detection-wave pulse generator 105 sets the detection-wave pulse transmission oscillator array Tx such that a detection wave that is a so-called plane wave for which detection-wave pulse transmission oscillators are driven in phase, passes through the entire region of interest roi. Preferably, the length a of the detection-wave pulse transmission oscillator array Tx is set larger than the region-of-interest width w. In the present example, the regimon-of-interest width w is set so as to be located inward by a predetermined distance β from the ends in the array direction of the detection-wave pulse transmission oscillator array Tx. Because the detection wave pw is a plane wave, the detection wave pw propagates in the Z direction vertical to the oscillator array direction. Therefore, the region of interest roi having a margin for the distance β at both ends in the X direction, is included in the ultrasonic irradiation region Ax. Thus, transmission and reception of a one-time detection wave enable generation of an acoustic line signal for the observation points in the entire region of interest roi, and additionally the detection-wave pulse pwpl can be transmitted such that the ultrasonic beam passes through the entire region of interest roi reliably. However, the number of times the detection wave is transmitted and received is not limited to the above. For example, a detection wave may be transmitted and received one time to generate an acoustic line signal for the observation points in part of the region of interest roi. The detection wave may be transmitted and received a plurality of times, and the acoustic line signals acquired from the respective pieces of transmission and reception may be combined to generate the acoustic line signal for the observation points in the entire region of interest roi.

The detection-wave pulse transmission oscillator array Tx may include all the plurality of oscillators 101a. The ultrasonic irradiation region Ax can include the maximum ultrasonic irradiation region Axmax of a plane wave.

Information indicating the detection-wave pulse transmission oscillator array Tx is output as a transmission control signal together with the pulse width of the detection-wave pulse pwpl, to the transmitter 106.

4. Transmitter 106

The transmitter 106 connected to the probe 101 through the multiplexer 107, includes a circuit that controls the timing of applying high voltage to each of the plurality of oscillators included in the push-wave transmission oscillator array Px or the detection-wave transmission oscillator array Tx corresponding to all or part of the plurality of oscillators 101a included in the probe 101 so that the probe 101 transmits an ultrasonic wave. Note that, as illustrated in FIG. 2, a push-wave pulse transmitter 1041 includes the push-wave pulse generator 104 and the transmitter 106, and a detection-wave pulse transmitter 1051 includes the transmitter 106 and the detection-wave pulse generator 105.

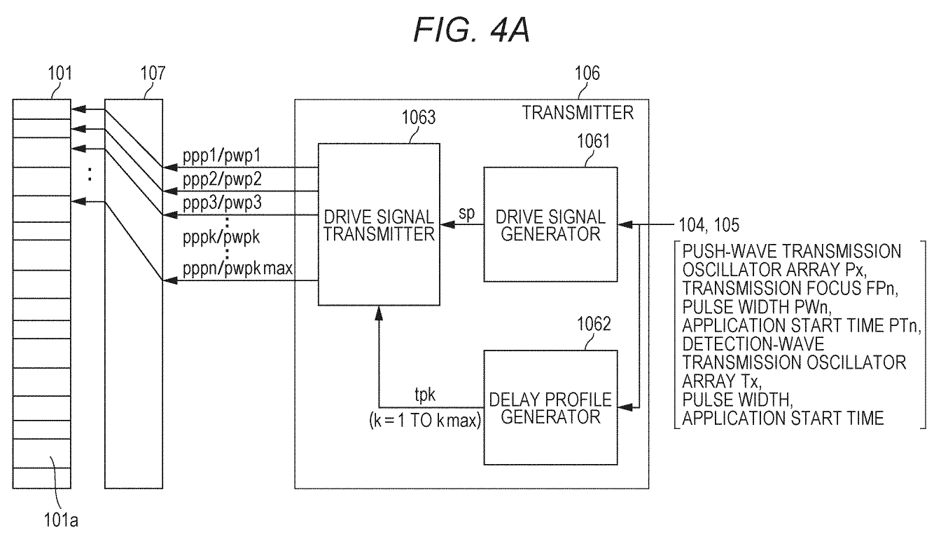

FIG. 4A is a functional block diagram of the configuration of the transmitter 106. As illustrated in FIG. 4A, the transmitter 106 includes a drive signal generator 1061, a delay profile generator 1062, and a drive signal transmitter 1063.

(1) Drive Signal Generator 1061

The drive signal generator 1061 includes a circuit that generates a pulse signal sp for transmission of the ultrasonic beam from the transmission oscillators corresponding to part or all of the oscillators 101a included in the probe 101, on the basis of information indicating the push-wave transmission oscillator array Px or the detection-wave transmission oscillator array Tx, information indicating the pulse width PWn and the application start time PTn of the push-wave pulse pppn, information indicating the pulse width and the application start time of the detection-wave pulse pwpl, in the transmission control signals from the push-wave pulse generator 104 and the detection-wave pulse generator 105.

(2) Delay Profile Generator 1062

The delay profile generator 1062 includes a circuit that sets and outputs, every oscillator, a delay time tpk (k is a natural number of 1 to kmax that is the number of oscillators 101a) from the application start time PTn for determination of the transmission timing of the ultrasonic beam, on the basis of information indicating the push-wave transmission oscillator array Pxn or the detection-wave transmission oscillator array Tx and the position of the transmission focus FPn, in the transmission control signals acquired from the push-wave pulse generator 104 and the detection-wave pulse generator 105. This arrangement delays transmission of the ultrasonic beam every oscillator by the delay time, to perform focusing of the ultrasonic beam.

(2) Drive Signal Transmitter 1063

The drive signal transmitter 1063 performs push-wave transmission processing of supplying the push-wave pulse ppp for transmission of the push wave, to each oscillator included in the push-wave transmission oscillator array Px in the plurality of oscillators 101a included in the probe 101, on the basis of the pulse signal sp from the drive signal generator 1061 and the delay time tpk from the delay profile generator 1062. The push-wave transmission oscillator array Px is selected by the multiplexer 107.

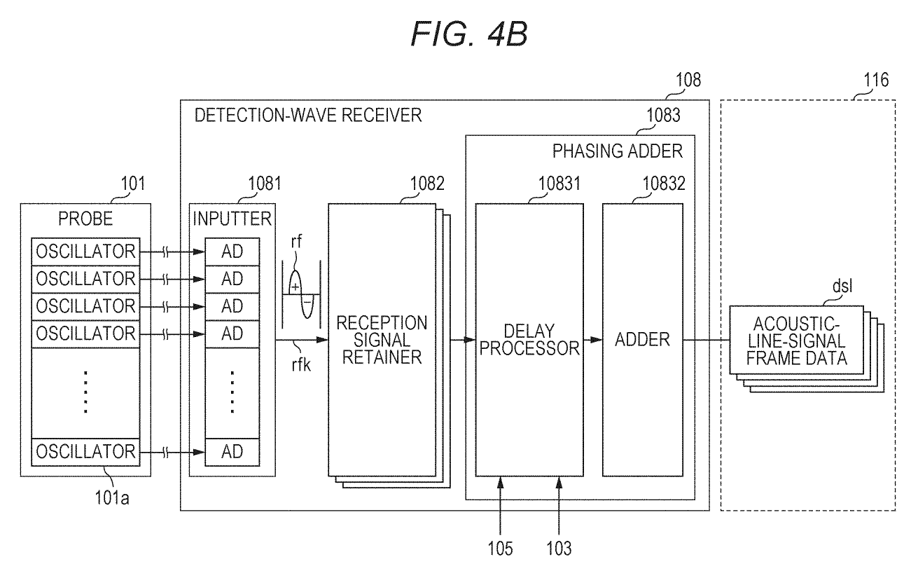

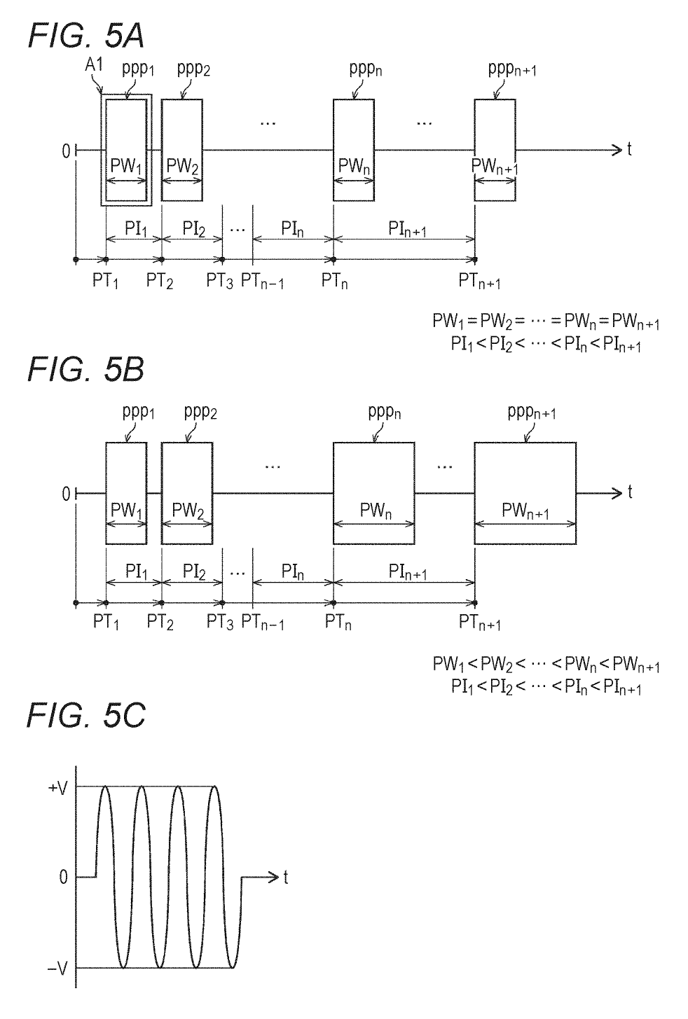

FIGS. 5A to 5C are schematic charts of the application timing of the push-wave pulse.

A push wave that causes physical displacement in a living body, requires remarkably larger power than a transmission pulse to be used for typical B-mode display does. That is, as drive voltage to be applied to a pulser (ultrasonic generator), typically a voltage of 30 to 40 V may be sufficient in acquisition of a B-mode image, but, for example, a voltage of 50 V or more is required for a push wave. Transmission pulse length is approximately several μsec in acquisition of a B-mode image, but a transmission pulse length of several hundred μsec is required for transmission of a push wave for one time.

In the present embodiment, as illustrated in FIG. 5A, the drive signal transmitter 1063 transmits the push-wave pulse pppn a plurality of times (nₘₐₓ) to the plurality of oscillators 101a at the application start time PTn. As illustrated in FIG. 5C, the push-wave pulse pppn having the predetermined pulse width PWn (time length), includes a burst signal having a predetermined amplitude in voltage (+V to −V) and a predetermined frequency. Specifically, the pulse width PWn may range, for example, from 100 to 200 μsec, the frequency may be, for example, 6 MHz, and the amplitude in voltage may range, for example, from +50 V to −50 V. However, needless to say, the application conditions are not limited to the above.

As illustrated in FIG. 5A, the application start time PTn every push-wave pulse pppn, has the time interval Pin of the application start time PTn every push-wave pulse pppn, increasing in descending order every application of the push-wave pulse pppn. This arrangement enables the depth-direction transmission focus position fin every push-wave pulse pppn, to have, as illustrated in FIG. 3A, a larger interval Δfzn between the adjacent transmission focuses fzn in the deeper portion of the object to be examined and to have a smaller interval Δfzn between the adjacent transmission focuses fzn in the shallower portion of the object to be examined.

As illustrated in FIG. 5A, the pulse width PWn every push-wave pulse pppn may be constant regardless of the application order of the push-wave pulse pppn.

Alternatively, as illustrated in FIG. 5B, the pulse width PWn every push-wave pulse pppn may increase in descending order every application of the push-wave pulse pppn. This arrangement compensates a reduction in the level of the shear wave due to propagation loss in transmission and reception at a deep portion, and retains the level of the shear wave uniformly between the shallow portion and the deep portion, so that measurement quality can improve. In comparison to the case of FIG. 5A, power distribution can be made properly so as to meet the time interval of the application start time PTn every push-wave pulse pppn.

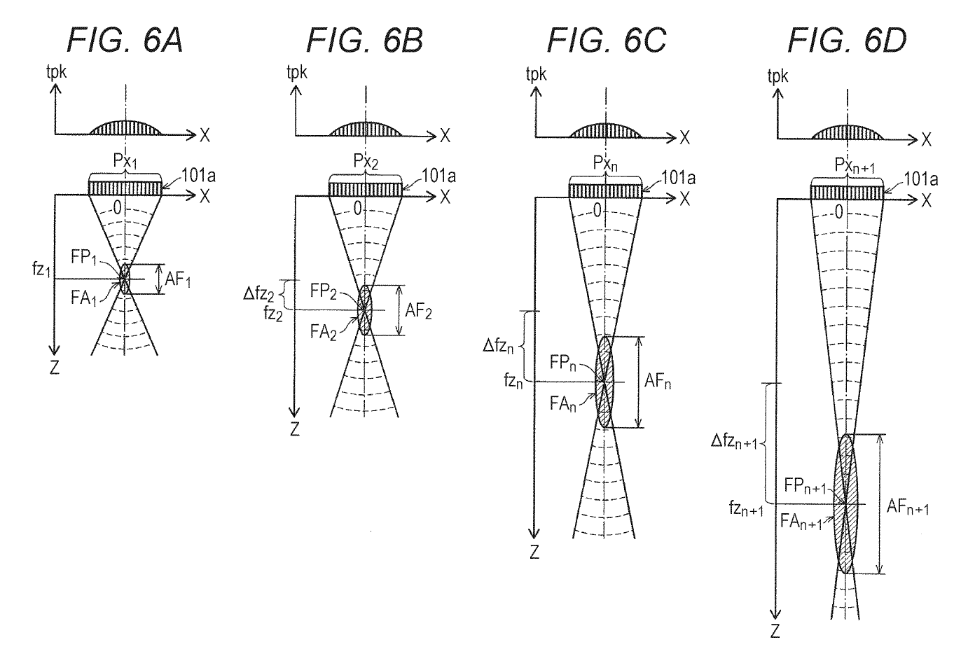

FIGS. 6A to 6D are schematic views of the overview of the push wave. The push-wave pulse ppp to which a distribution having a large delay time tpk to an oscillator located at the center of the oscillator array is applied, is transmitted to the push-wave transmission oscillator array Px at the application start time PTn every push-wave pulse pppn. This arrangement allows the push-wave transmission oscillator array Px to transmit the push wave ppn including the ultrasonic beam to focus onto the specific part in the object to be examined corresponding to the transmission focus FPn. At this time, the push wave ppn is transmitted such that the interval Δfzn between the adjacent transmission focuses fzn is larger in the deeper portion of the object to be examined and is smaller in the shallower portion of the object to be examined.

When a region in which the transmission focus FP is located and the energy density of the ultrasonic beam is a predetermined value or more, is defined as a focus region FAn to the transmission focus FPn that is the center of the ultrasonic beam at the depth at which the ultrasonic beam focuses, the length AFn in the depth direction of the focus region FAn is longer in the deeper portion of the object to be examined and is shorter in the shallower portion of the object to be examined.

For example, the length APn in the depth direction of the focus region FAn may be prescribed by the following expression.

$$\begin{matrix}[ Mathematical Formula 1 ] & \\ {AF}_{n + 1} = {AF}_{n} \times \frac{{fz}_{n + 1}}{{AF}_{1}} & ( Expression 1 )\end{matrix}$$

The coordinate in the depth direction of the transmission focus fzn may be prescribed by the following expression when β is a coefficient.

$$\begin{matrix}[ Mathematical Formula 2 ] & \\ {fz}_{n + 1} = \frac{2 {fz}_{1} + β {a}_{1}}{2 {fz}_{1} - β {a}_{1}} {fz}_{n} \\ ( β \text{:} coefficient ) & ( Expression 2 )\end{matrix}$$

The drive signal transmitter 1063 performs detection-wave transmission processing of supplying the detection-wave pulse pwpl for transmission of the ultrasonic beam, to each oscillator included in the detection-wave transmission oscillator array Tx in the plurality of oscillators 101a included in the probe 101. The detection-wave transmission oscillator array Tx is selected by the multiplexer 107. However, the configuration according to the supply of the detection-wave pulse pwpl is not limited to the above, and thus, for example, no multiplexer 107 may be provided.

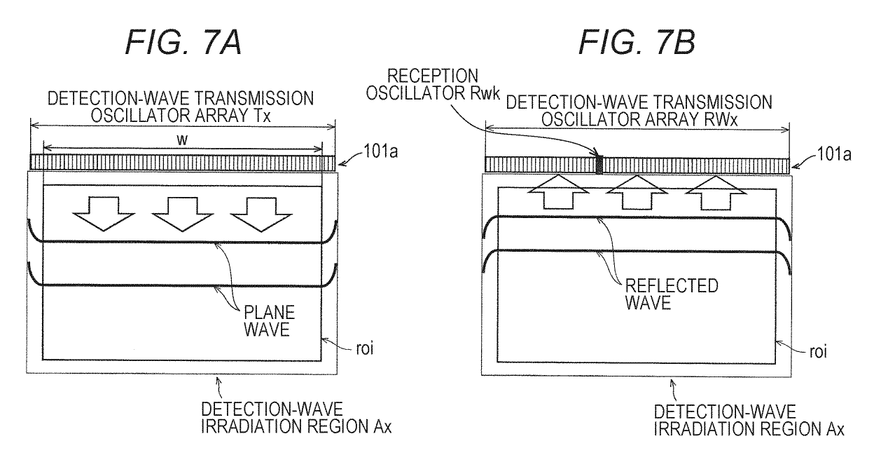

FIG. 7A is a schematic view of the overview of detection-wave transmission. No delay time tpk is applied to the oscillators included in the detection-wave transmission oscillator array Tx, and the detection-wave pulse pwpl in phase is transmitted to the detection-wave transmission oscillator array Tx. This arrangement allows, as illustrated in FIG. 7A, each oscillator in the detection-wave transmission oscillator array Tx to propagate the plane wave to travel in the depth direction of the object to be examined. The region in a plane corresponding to the range in the object to be examined at which the detection wave arrives, the plane including the detection-wave transmission oscillator array Tx, is the detection-wave irradiation region Ax.

After the transmission of the push-wave pulse ppp, the transmitter 106 transmits the detection-wave pulse pwpl a plurality of times, on the basis of the transmission control signal from the detection-wave pulse generator 105. After the transmission of the push-wave pulse ppp for one time, transmission for each time in a flow of transmission of the detection-wave pulse pwpl in which the same detection-wave transmission oscillator array Tx transmits the detection-wave pulse pwpl a plurality of times, is referred to as a “transmission event”.

2. Configuration of Detection-Wave Receiver 108

The detection-wave receiver 108 includes a circuit that generates the acoustic line signal corresponding to a plurality of observation points Pij in the detection-wave irradiation region Ax, to generate a sequence of acoustic-line-signal frame data dsl (l is a natural number of 1 to m, but the acoustic-line-signal frame data dsl is provided in a case where the numbers are not distinguished), on the basis of the reflected wave from the tissue of the object to be examined received on a time series basis in the plurality of oscillators 101a, corresponding to the detection-wave pulse pwpl for each time. That is, after the transmission of the detection-wave pulse pwpl, the detection-wave receiver 108 generates the acoustic line signal from the electric signal acquired by the plurality of oscillators 101a on the basis of the reflected wave received by the probe 101. Here, i represents a natural number indicating the coordinate in the x direction in the detection-wave irradiation region Ax, and j represents a natural number indicating the coordinate in the z direction in the detection-wave irradiation region Ax. Note that the “acoustic line signal” is a signal including a reception signal (RP signal) subjected to phasing and adding processing.

FIG. 4B is a functional block diagram of the configuration of the detection-wave receiver 108. The detection-wave receiver 108 includes an inputter 1081, a reception signal retainer 1082, and a phasing adder 1083.

2.1 Inputter 1081

The inputter 1081 connected to the probe 101 through the multiplexer 107, includes a circuit that generates a reception signal (RF signal) on the basis of a reflected wave in the probe 101. Here, the reception signal rfk (k is a natural number of 1 to n) includes a so-called RF signal A/D converted from the electric signal converted from the reflected wave received by each oscillator on the basis of the transmission of the detection-wave pulse pwpl. The reception signal rfk includes an array of signals (reception signal array) in the transmission direction of the ultrasonic wave (depth direction of the object to be examined), received by each reception oscillator rwk.

The inputter 1081 generates the reception signal rfk including an array to each reception oscillator rwk every transmission event, on the basis of the reflected wave acquired by each reception oscillator rwk. A reception oscillator array includes an oscillator array corresponding to part or all of the plurality of oscillators 101a included in the probe 101, and is selected by the multiplexer 107 on the basis of an instruction from the controller 116. In the present example, all the plurality of oscillators 101a is selected as the reception oscillator array. This arrangement enables, as illustrated in FIG. 7B illustrating the overview of reflected detection-wave reception, generation of the reception oscillator array to all the oscillators such that all the oscillators receive the reflected wave from the observation points included in the entire region in the detection-wave irradiation region Ax, in reception processing for one time. The generated reception signal rfk is output to the reception signal retainer 1082.

2.2 Reception Signal Retainer 1082

The reception signal retainer 1082 including a computer-readable recording medium, can adopt, for example, a semiconductor memory. The reception signal retainer 1082 inputs the reception signal rfk to each reception oscillator rwk from the inputter 1081 in synchronization with the transmission event, to retain the reception signal rfk until one piece of acoustic-line-signal frame data is generated.

Note that the reception signal retainer 1082 may be part of the data storage 115.

2.3 Phasing Adder 1083

The phasing adder 1083 includes a circuit that performs delay processing to the reception signal rfk received by a reception oscillator Rpk included in a detection-wave pulse reception oscillator array Rx, from the observation point Pij in the region of interest roi in synchronization with the transmission event, and then performs addition to all reception oscillators Rpk, to generate the acoustic line signal ds. The detection-wave pulse reception oscillator array Rx including the reception oscillators Rpk corresponding to part or all of the plurality of oscillators 101a included in the probe 101, is selected by the phasing adder 1083 and the multiplexer 107 on the basis of an instruction from the controller 116. In the present example, as the detection-wave pulse reception oscillator array Rx, an oscillator array including at least all of the oscillators included in the detection-wave pulse transmission oscillator array Tx in each transmission event, is selected.

The phasing adder 1083 includes a delay processor 10831 that performs processing to the reception signal rfk, and an adder 10832.

(1) Delay Processor 10831

The delay processor 10831 includes a circuit that performs compensation with a reflected ultrasonic arrival time difference (amount of delay) to each reception oscillator Rpk, in which the difference in distance between the observation point Pij and each reception oscillator Rpk is divided by a sonic value, from the reception signal rfk to the reception oscillator Rpk in the detection-wave pulse reception oscillator array Rx, and identifies a reception signal corresponding to the reception oscillator Rpk, based on the reflected ultrasonic wave from the observation point Pij.

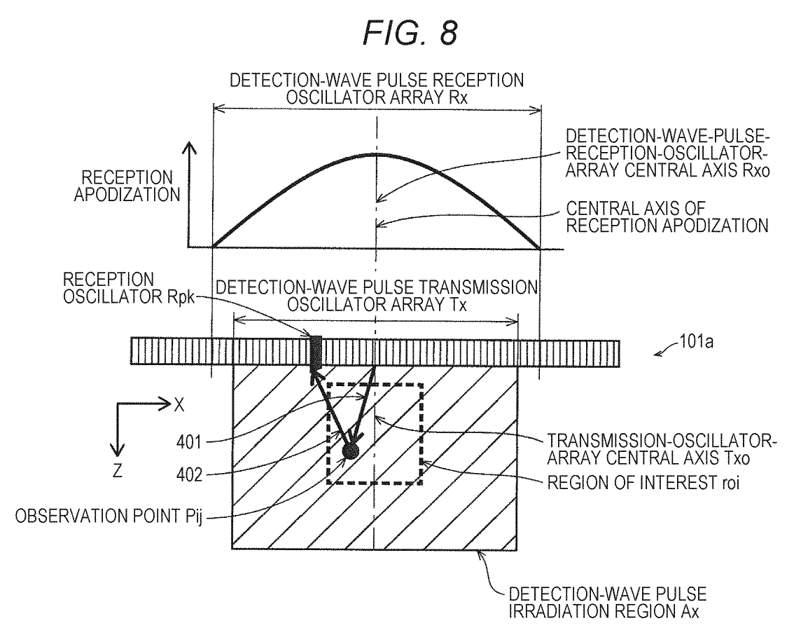

FIG. 8 is a schematic view of the overview of a method of calculating the propagation path of an ultrasonic wave in the delay processor 10831. FIG. 8 illustrates a propagation path in which the ultrasonic wave radiated from the detection-wave pulse transmission oscillator array Tx reflects at the observation point Pij at an arbitrary point in the region of interest roi, to arrive at the reception oscillator Rpk.

a) Calculation of Transmission Time

As described above, the detection wave pwl to be transmitted from the detection-wave transmission oscillator array Tx (entire oscillator array (101a)) is a plane wave. Therefore, corresponding to the transmission event, the delay processor 10831 calculates a transmission path to the observation point Pij as a shortest path 4101 in which the detection wave pwl generated from the detection-wave transmission oscillator array Tx arrives at the observation point Pij, and then makes the shortest path 401 divided by the speed of sound to calculate transmission time.

b) Calculation of Reception Time

Corresponding to the transmission event, the delay processor 10831 calculates, for the observation point Pij, a reception path in which the detection wave pwl reflected at the observation point Pij arrives at the reception oscillator Rpk included in the detection-wave reception oscillator array Rx. For the reception path in which the reflected wave from the observation point Pij returns to the reception oscillator Rpk, the length of a path 402 from the arbitrary observation point Pij to each reception oscillator Rpk is calculated geometrically. The length of the path 402 is divided by the speed of sound to calculate reception time.

c) Calculation of Amount of Delay

Next, the delay processor 10831 calculates total propagation time to each reception oscillator Rpk from the transmission time and the reception time, to calculate the amount of delay to each reception oscillator Rpk, to be applied to the reception signal array rfk, on the basis of the total propagation time.

d) Delay Processing

Next, the delay processor 10831 identifies, as a signal corresponding to the reception oscillator Rpk based on the reflected wave from the observation point Pij, the reception signal rfk corresponding to the amount of delay (reception signal corresponding to time from which the amount of delay is subtracted) from the reception signal array rfk to each reception oscillator Rpk.

Corresponding to the transmission event, the delay processor 10831 identifies, with the reception signal rfk from the reception signal retainer 1082 as an input, the reception signal rfk to each reception oscillator Rpk for all the observation points Pij located in the region of interest roi.

(2) Adder 10832

The adder 10832 includes a circuit that performs addition with the reception signal rfk identified corresponding to the reception oscillator Rpk, output from the delay processor 10831 as an input, to generate an acoustic line signal dsij phased and added to the observation Pij.

Furthermore, addition may be performed to the reception signal rfk identified corresponding to each reception oscillator Rpk, multiplied by reception apodization (weighting progression), to generate the acoustic line signal dsij to the observation point Pij. The reception apodization is a weighting-factor progression to be applied to a reception signal corresponding to the reception oscillator Rpk in the detection-wave reception oscillator array Rx. The reception apodization is set such that a weight is maximum to the oscillator located at the center in the array direction of the detection-wave reception oscillator array Rx. The central axis of the distribution of the reception apodization agrees with a detection-wave-reception-oscillator-array central axis Rxo. The distribution has a symmetrical shape with respect to the central axis. The distribution is not particularly limited in shape.

The adder 10832 generates the acoustic line signal dsij for all the observation points Pij included in the region of interest roi, to generate the acoustic-line-signal frame data dsl.

Then, transmission and reception of the detection-wave pulse pwpl are repeated in synchronization with the transmission event, to generate the acoustic-line-signal frame data dsl to all the transmission events. The generated acoustic-line-signal frame data dsl is output to and is stored in the data storage 115 every transmission event.

3. Displacement Detector 109

The displacement detector 109 includes a circuit that detects displacement of the tissue in the detection-wave irradiation region Ax, from the sequence of acoustic-line-signal frame data dsl.

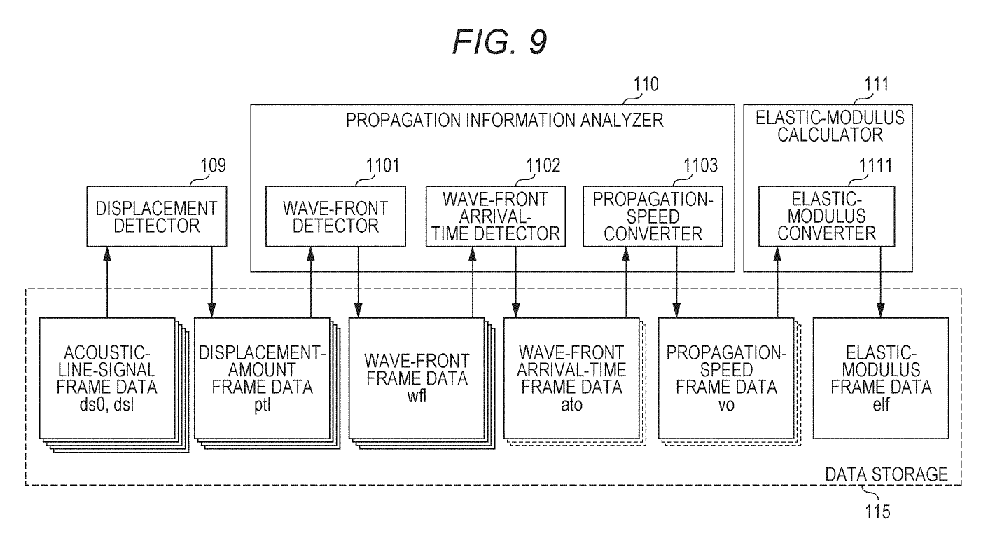

FIG. 9 is a functional block diagram of the configurations of the displacement detector 109, the propagation information analyzer 110, and the elastic-modulus calculator 111.

The displacement detector 109 acquires one frame of acoustic-line-signal frame data dsl to be detected for displacement included in the sequence of acoustic-line-signal frame data dsl and one frame of acoustic-line-signal frame data ds0 for reference (hereinafter, referred to as “reference acoustic-line-signal frame data ds0”), from the data storage 115 through the controller 116. The reference acoustic-line-signal frame data ds0 is a reference signal for extracting displacement with the shear wave in the acoustic-line-signal frame data dsl corresponding to each transmission event, and is specifically acoustic-line-signal frame data acquired from the detection-wave irradiation region Ax before the transmission of the push-wave pulse ppp. Then, the displacement detector 109 detects displacement (motion in image information) ptij of the observation point Pij in the detection-wave irradiation region Ax of the acoustic-line-signal frame data dsl, from the difference between the acoustic-line-signal frame data dsl and the reference acoustic-line-signal frame data ds0, to generate displacement-amount frame data pt (I is a natural number of 1 to m, but the displacement-amount frame data pd is provided in a case where the numbers are not distinguished) with the displacement ptij in association with the coordinates of the observation point Pij. The displacement detector 109 outputs the generated displacement-amount frame data ptl to the data storage 115.

4. Propagation Information Analyzer 110

The propagation information analyzer 110 includes a circuit that calculates wave front arrival-time data atij for the plurality of observation points Pij in the region of interest roi, to calculate wave-front arrival-time frame data atl to the region of interest roi. The propagation information analyzer 110 includes a wave-front detector 1101, a wave-front arrival-time detector 1102, and a propagation-speed converter 1103.

The wave-front detector 1101 generates a sequence of wave-front frame data wfl (t is a natural number of 1 to m, but the wave-front frame data wfl is provided in a case where the numbers are not distinguished) expressing the wave-front position wfij of the shear wave at a plurality of points in time on the temporal axis, corresponding to the detection-wave pulse pwpl for each lime, for the observation point Pij in the region of interest roi, from a sequence of displacement-amount frame data p11 every transmission event, and outputs the sequence of wave-front frame data wfl to the data storage 115.

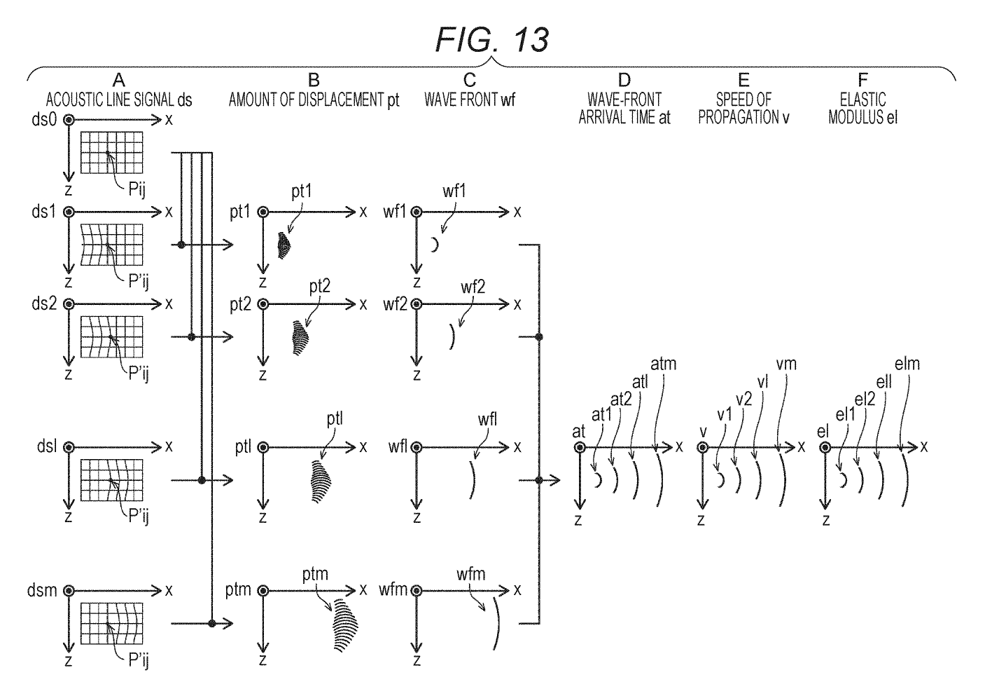

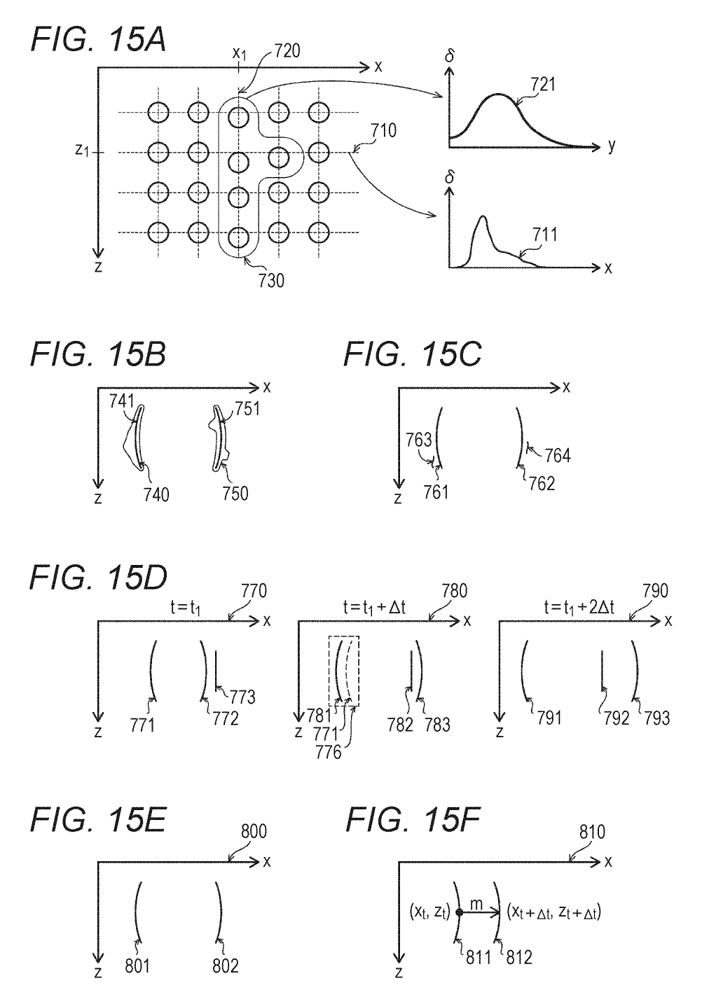

Specifically, the wave-front detector 1101 acquires the displacement-amount frame data ptl from the data storage 115. The propagation information analyzer 110 detects local maximum points of the displacement data ptij from the displacement-amount frame data ptl, to extract the coordinates ij of the local maximum points of the displacement data ptij in succession in the XZ plane as the wave-front position wfij of the shear wave. A method of extracting the wave-front position wfij from the displacement data ptij, will be described later in FIGS. 13, 15A, and 15B. The wave-front position wfij is extracted for the region of interest roi in each transmission event, to generate the sequence of wave-front frame data wfl.

The wave-front arrival-time detector 1102 detects the wave front wf of the shear wave, the position wfij, and the traveling direction thereof at the time at which the displacement-amount frame data p11 based on the wave-front frame data wfl is acquired, from the sequence of wave-front frame data wfl, generates wave-front arrival-time frame data ato (o is a natural number expressing the number of different wave fronts, but wave-front arrival-time frame data at is provided in a case where the numbers are not distinguished), on the basis of the wave-front position wfij of the wave-front frame data wfl and frame-data acquisition time tl, and outputs the wave-front arrival-time frame data ato to the data storage 115.

The propagation-speed converter 1103 converts the wave-front arrival-time frame data ato, into propagation speed data vij at the observation point Pij in the region of interest roi, generates propagation-speed frame data vo (o is a natural number expressing the number of different wave fronts, but propagation-speed frame data v is provided in a case where the numbers are not distinguished) to the region of interest roi, and outputs the propagation-speed frame data vo to the data storage 115.

6. Elastic-Modulus Calculator 111

The elastic-modulus calculator 111 includes a circuit that calculates the elastic modulus of the tissue for the observation point Pij in the region of interest roi, to calculate elastic-modulus frame data elf to the region of interest roi. The elastic-modulus calculator 111 includes an elastic-modulus converter 1111. With the propagation-speed frame data vo as an input, the elastic-modulus converter 1111 converts the propagation-speed frame data v into elastic-modulus data el at the observation point Pij in the region of interest roi, generates the elastic-modulus frame data elf to the region of interest roi, and outputs the elastic-modulus frame data elf to the data storage 115.

8. Other Configurations

The data storage 115 includes a recording medium that sequentially records the reception signal array rf, the sequence of acoustic-line-signal frame data ds), the sequence of displacement-amount frame data ptl, the sequence of wave-front frame data wfl, the wave-front arrival-time frame data at, compensated wave-front arrival-time frame data cat, the propagation-speed frame data vl, and the elastic-modulus frame data elf that are generated.

The controller 116 controls each block in the ultrasonic diagnostic apparatus 100, on the basis of a command from the operation inputter 102. The controller 116 can adopt a processor, such as a CPU.

Although not illustrated, the ultrasonic diagnostic apparatus 100 includes a B-mode image generator that generates an ultrasonic image (B-mode image) on a time series basis, on the basis of the reflected component from the tissue of the object to be examined, in the acoustic line signal output on the basis of the transmission and reception of the detection wave performed in the transmitter 106 and the detection-wave receiver 108, with no transmission of the push-wave pulse ppp. The B-mode image generator inputs the acoustic-line-signal frame data from the data storage 115, performs processing, such as envelop detection and logarithm compression, to the acoustic line signal, converts the acoustic line signal into a brightness signal corresponding to the intensity thereof, performs coordinate transformation to the brightness signal into a Cartesian coordinate system, and generates B-mode-image frame data. Note that the ultrasonic transmission and reception in the transmitter 106 and the detection-wave receiver 108 for acquisition of the acoustic line signal for B-mode image generation, can adopt a publicly known method. The generated B-mode-image frame data is output to and is stored in the data storage 115. The display controller 113 arranges the B-mode image as a display image and causes the display 114 to display the B-mode image.

The elastic-modulus calculator 111 may generate and display an elasticity image on which color information is mapped, on the basis of the elastic modulus expressed by the elastic-modulus frame data elf. For example, an elasticity image color-coded in red at the coordinates at which the elastic modulus is an certain value or more, in green at the coordinates at which the elastic modulus is less than the certain value, and in black at the coordinates at which no elastic modulus can be acquired, may be generated. Thus, the convenience of the operator can improve. The elastic-modulus calculator 111 outputs the elastic-modulus frame data elf and the elasticity image that are generated, to the data storage 115, and the controller 116 outputs the elasticity image to the display controller 113. Furthermore, the display controller 113 may perform geometric transformation such that the elasticity image is rendered in image data for screen display, and may output the elasticity image after the geometric transformation to the display 114.

<Operation>

The operation of the integrated SWS sequence of the ultrasonic diagnostic apparatus 100 having the configuration, will be described.

1. Overview of Operation

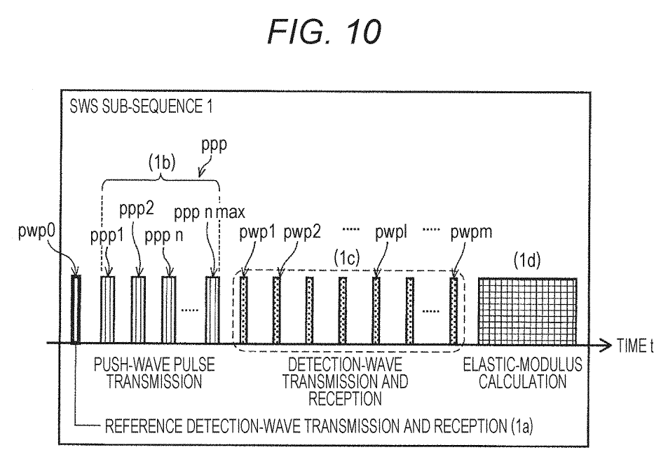

FIG. 10 is a schematic view of the overview of an integrated SWS sequence process in the ultrasonic diagnostic apparatus 100. The SWS sequence in the ultrasonic diagnostic apparatus 100 includes: a process (la) of performing reference detection-wave transmission and reception and acquiring the reference acoustic-line-signal frame data ds0 for extracting displacement with the shear wave corresponding to each transmission event after that; a process (1b) of transmitting the push-wave pulse pppn (n=1 to nₘₐₓ) a plurality of times (nₘₐₓ), transmitting the push wave ppn to focus onto the specific part FP in the object to be examined, a plurality of times (nₘₐₓ), and causing the shear-wave excitation in the object to be examined; a process (1c) of performing the transmission and reception of the detection-wave pulse pwpl so that the detection wave pwl that passes through the region of interest roi is repeatedly transmitted and received a plurality of times (m); and an elastic-modulus calculation process (1d) of performing the shear-wave propagation analysis and calculating the propagation speed vf of the shear wave and the elastic modulus elf.

2. Operation of SWS Sequence

The operation of ultrasonic elastic-modulus measurement processing after the display 114 displays the B-mode image in which the tissue is drawn on the basis of the reflected component from the tissue of the object to be examined on the basis of a publicly known method, will be described below.

Note that, for the B-mode-image frame data, the acoustic-line-signal frame data is generated on a time series basis on the basis of the reflected component from the tissue of the object to be examined on the basis of the transmission and reception of the ultrasonic wave performed in the transmitter 106 and the detection-wave receiver 108, with no transmission of the push-wave pulse ppp. The acoustic line signal is subjected to the processing such as envelop detection and logarithm compression, so as to be converted into the brightness signal. After that, the brightness signal is subjected to the coordinate transformation into the Cartesian coordinate system, to generate the B-mode-image frame data. The display controller 113 causes the display 114 to display the B-mode image in which the tissue of the object to be examined is drawn.

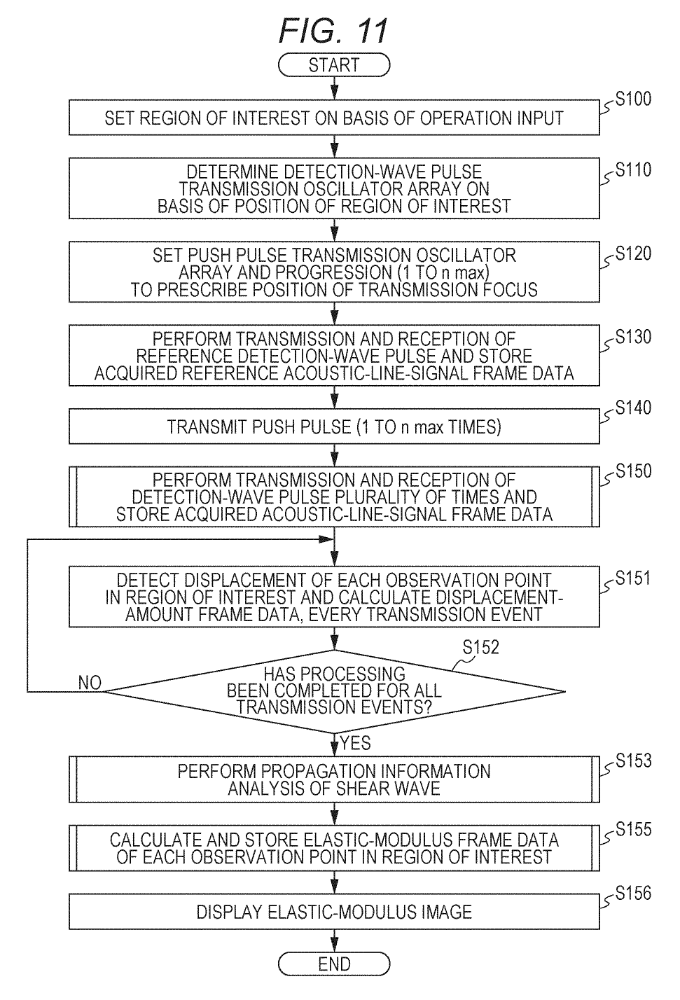

FIG. 11 is a flowchart of the operation of ultrasonic elastic-modulus calculation in the ultrasonic diagnostic apparatus 100.

[Steps S00 to S140]

At step S100, with the display 114 displaying the B-mode image that is a tomographic image of the object to be examined in real time acquired by the probe 101, the region-of-interest setter 103 sets the region of interest roi expressing the range to be analyzed in the object to be examined, with respect to the position of the probe 101, with the information specified by the operator as an input from the operation inputter 102, and outputs the information to the controller 116.

For the specification of the region of interest roi by the operator, for example, while the display 114 is displaying the latest B-mode image recorded in the data storage 115, the region of interest roi is specified through an inputter (not illustrated), such as a touch panel or a mouse. Note that, for the region of interest roi, for example, the entire region of the B-mode image may be specified as the region of interest roi or a certain range including the central portion of the B-mode image may be specified.

At step S120, the push-wave pulse generator 104 inputs the information indicating the region of interest roi from the controller 116, and sets the position of the transmission focus FPn of the push-wave pulse pppn (n=1 to nₘₐₓ) and the push-wave transmission oscillator array Pxn. In the present example, as illustrated in FIG. 3A, the push-wave transmission oscillator array Pxn is constant regardless of the transmission order n of the push wave, and includes all the plurality of oscillators 101a. The array-direction transmission focus position fx agrees with the array-direction central position we of the region of interest roi. The depth-direction transmission focus position fzn (n=1 to nₘₐₓ) has a larger interval Δfzn between the adjacent transmission focuses fzn in the deeper portion of the object to be examined, and has a smaller interval Δfzn between the adjacent transmission focuses fzn in the shallower portion of the object to be examined. However, the positional relationship between the detection-wave irradiation region Ax and the transmission focus FP is not limited to the above, and thus may be appropriately changed depending on a mode of a pan to be examined in the object to be examined.

The information indicating the position of the transmission focus FP and the push-wave transmission oscillator array Px, is output as the transmission control signal together with the pulse width PWn and the application start time PTn of the push-wave pulse ppp, to the transmitter 106.

At step S130, the transmitter 106 transmits the detection-wave pulse pwp0 to the oscillators included in the detection-wave transmission oscillator array Tx, to cause transmission of the detection wave pw0 into the object to be examined. The detection-wave receiver 108 receives the reflected wave ec of the detection wave pw0, to generate the reference acoustic-line-signal frame data ds0 that is the reference for displacement of the tissue. The reference acoustic-line-signal frame data ds0 is output to and is stored in the data storage 115. A method of generating the acoustic-line-signal frame data, will be described later.

At step S140, the transmitter 106 transmits the push-wave pulse pppn a plurality of times (nₘₐₓ) to the oscillators included in the push-wave transmission oscillator array Pxn, to cause the oscillators to transmit, a plurality of times (nₘₐₓ), the push wave ppn including the ultrasonic beam to focus onto the specific part in the object to be examined corresponding to the transmission focus FP.

Specifically, the transmitter 106 generates a transmission profile, on the basis of the transmission control signal including the information indicating the position of the transmission focus FPn and the push-wave transmission oscillator array Pxn, and the pulse width PWn and the application start time PTn of the push-wave pulse pppn, acquired from the push-wave pulse generator 104. The transmission profile includes the pulse signal sp and the delay time tpk to each transmission oscillator included in the push-wave transmission oscillator array Pxn. Then, the push-wave pulse pppn is supplied to each transmission oscillator, on the basis of the transmission profile. Each transmission oscillator transmits the push wave ppn to focus onto the specific part of the object to be examined. The transmitter 106 performs the operation a plurality of times (nₘₐₓ).

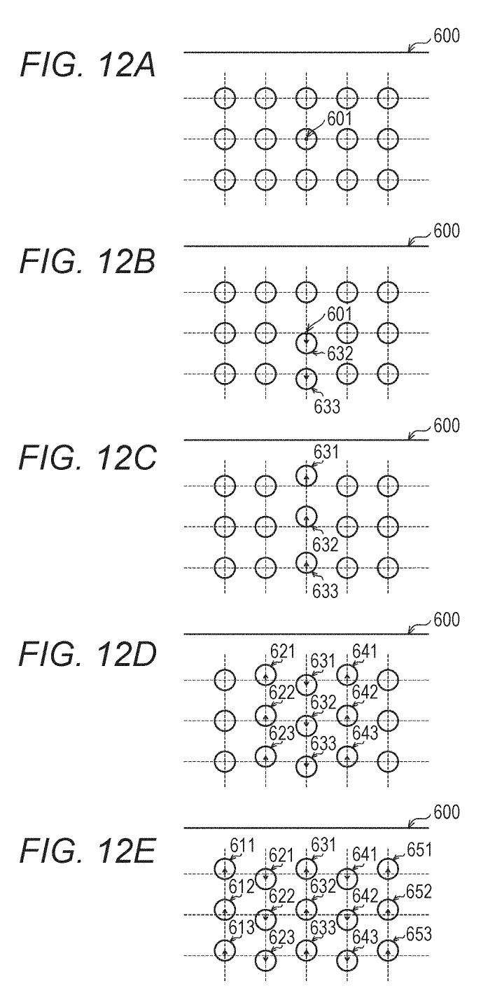

Here, generation of the shear wave with the push wave pp will be described with the schematic views of FIGS. 12A to 12E. FIGS. 12A to 12E are schematic views of the state of the generation of the shear wave with the push wave pp. FIG. 12A is a schematic view of the tissue, before the push wave pp is applied, in a region in the object to be examined corresponding to the detection-wave irradiation region Ax. In FIGS. 12A to 12F, each individual “c” indicates part of the tissue in the object to be examined, and each intersection of broken lines indicates the central position of the tissue “o” in a case where no load is present.

Here, when the push wave pp is applied to a focal part 601 in the object to be examined corresponding to the transmission focus FP, with the probe 101 in close contact with a skin surface 600, as illustrated in the schematic view of FIG. 12B, tissue 632 located at the focal part 601 is pushed to move in the traveling direction of the push wave pp. Tissue 633 located in the traveling direction of the push wave pp with respect to the tissue 632, is pushed by the tissue 632, to move in the traveling direction of the push wave pp.

Next, because the tissues 632 and 633 attempt to restore the respective original positions when the transmission of the push wave pp finishes, as illustrated in the schematic view of FIG. 12C, tissues 631 to 633 start to oscillate along the traveling direction of the push wave pp.

Then, as illustrated in the schematic view of FIG. 12D, the oscillation propagates to tissues 621 to 623 and tissues 641 to 643 adjacent to the tissues 631 to 633.

Furthermore, as illustrated in the schematic view of FIG. 12E, the oscillation further propagates to tissues 611 to 613 and tissues 651 to 653. Therefore, the oscillation propagates in the direction orthogonal to the oscillating direction, in the object to be examined. That is the shear wave occurs at the position at which the push wave pp is applied, and propagates in the object to be examined.

[Step S150]

Referring back to FIG. 11, the description will be continued.

At step S150, transmission and reception of the detection-wave pulse pwpl are performed a plurality of times for the region of interest roi, and the acquired sequence of acoustic-line-signal frame data dsl is stored. Specifically, the transmitter 106 transmits the detection-wave pulse pwpl to the oscillators included in the detection-wave transmission oscillator array Tx for the object to be examined, and the detection-wave receiver 108 generates the acoustic-line-signal frame data dsl, on the basis of the reflected wave ec received by the oscillators included in the detection-wave pulse reception oscillator array Rx. Immediately after transmission of the last push wave ppnₘₐₓ finishes, for example, the processing is repeatedly performed ten thousand times per second. This arrangement allows repeated generation of the acoustic-line-signal frame data dsl in the detection-wave irradiation region Ax of the object to be examined until the propagation finishes immediately after the shear wave occurs. The generated sequence of acoustic-line-signal frame data dsl is output to and is stored in the data storage 115.

The details of a method of generating the acoustic-line-signal frame data dsl at step S150, will be described later.

[Step S151]

At step S151, the displacement detector 109 detects displacement at the observation point Pij in the region of interest roi in each transmission event.

FIG. 13 is a schematic view of the operation of displacement detection and shear-wave propagation analysis.

First, the displacement detector 109 acquires the reference acoustic-line-signal frame data ds0 stored in the data storage 115 at step S130. As described above, the reference acoustic-line-signal frame data ds0 is acoustic-line-signal frame data acquired before the transmission of the push wave pp, namely, before the shear wave occurs.

Next, from the difference between the reference acoustic-line-signal frame data ds0 and each piece of acoustic-line-signal frame data dsl stored in the data storage 115 at step S150, the displacement detector 109 detects displacement of each pixel at the time at which the acoustic-line-signal frame data dsl is acquired.

Array A in FIG. 13 indicates the reference acoustic-line-signal frame data ds0 and the acoustic-line-signal frame data dsl generated in each transmission event. Array B indicates the displacement-amount frame data ptl calculated for each transmission event at step S150. As illustrated in Array A and Array B of FIG. 13, it is detected which acoustic line signal dsij of the observation point P′ij in the acoustic-line-signal frame data dsl is similar to the acoustic line signal dsij of the observation point Pij in the reference acoustic-line-signal frame data ds0, in comparison between the acoustic-line-signal frame data dsl and the reference acoustic-line-signal frame data ds0. Then, the positional variation of the observation point P′ij to the observation point Pij is calculated to detect the displacement-amount frame data ptl.

Specifically, for example, the acoustic-line-signal frame data dsl is divided into regions each having a predetermined size, such as 8 pixels×8 pixels, and pattern matching is performed between each region and the reference acoustic-line-signal frame data ds0. Then, the displacement of each pixel in the acoustic-line-signal frame data dsl is detected.

As a pattern matching method, for example, there can be provided a method including: calculating, between each region and the same-size reference region in the reference acoustic-line-signal frame data ds0, a difference in brightness value every corresponding pixel; calculating the total value of the absolute values of the differences; regarding, for a combination of the region in which the total value is minimum and the reference region, the region and the reference region as the same region; and detecting the distance between the reference point of the region (e.g., the upper-left corner) and the reference point of the reference region, as the displacement.

Note that the region size does not necessarily have 8 pixels×8 pixels, and, for example, the total value of the squared differences in brightness value may be used instead of the total value of the absolute values of the differences in brightness value. As the displacement, the difference in the y coordinate (difference in depth) between the reference point in the region and the reference point in the reference region, may be calculated. This arrangement allows, for the displacement, calculation of how much the tissue of the object to be examined corresponding to each observation point Pij in each piece of acoustic-line-signal frame data dsl, has moved due to the push wave pp or the shear wave.

Note that the method of detecting the displacement is not limited to the pattern matching. For example, any technique of detecting the amount of movement between two pieces of acoustic-line-signal frame data dsl, may be used, such as correlation processing between the acoustic-line-signal frame data dsl and the reference acoustic-line-signal frame data ds0. The displacement detector 109 associates the displacement of each observation point Pij according to one frame of acoustic-line-signal frame data dsl, with the coordinates ij of the observation point, generates the displacement-amount data ptij of the observation points in the region of interest roi, and outputs the generated displacement-amount frame data ptl for the region of interest roi, to the data storage 115.

[Steps S152 to S155]

The propagation information analyzer 110 outputs and stores the generated displacement-amount frame data ptl into the data storage 115 (step S173). It is determined whether the processing at step S151 has been completed for all the prescribed transmission events (step S152). In a case where the completion has not been made, the processing goes back to step S151 and a flow of processing is performed for the transmission event of the next detection-wave pulse pwpl. In a case where the completion has been made, the processing proceeds to step S153.

At step S153, with the sequence of displacement-amount frame data ptl as an input, the propagation information analyzer 110 detects the wave front of the shear wave from the displacement-amount data ptij of the observation point Pij in the region of interest roi in each transmission event, to generate the sequence of wave-front frame data wfl expressing the wave-front position wfij. Furthermore, the propagation information analyzer 110 detects the wave front wf and the position wfij of the shear wave at the time at which the displacement-amount frame data ptl based on the frame data is acquired, from the sequence of wave-front frame data wfl, generates the wave-front arrival-time frame data ato every wave front, and outputs the wave-front arrival-time frame data ato to the data storage 115. Furthermore, the propagation information analyzer 110 calculates the propagation speed data vij of the shear wave for the observation point Pij in the region of interest roi, and outputs the propagation speed data vij of the shear wave to the data storage 115. The details of a method of performing the propagation information analysis of the shear wave at step S153, will be described.

At step S155, the elastic-modulus calculator 111 calculates the elastic-modulus data elij for the observation point Pij in the region of interest roi, calculates the elastic-modulus frame data elf to the region of interest roi, and outputs the elastic-modulus frame data elf to the data storage 115. The details of a method of calculating the elastic-modulus frame data elf at step S155, will be described later.

At step S156, the elastic-modulus calculator 111 generates the elasticity image on which color information is mapped, on the basis of the elastic modulus expressed by the elastic-modulus frame data elf. The display controller 113 performs the geometric transformation such that the elasticity image is rendered in the image data for screen display, and outputs the elasticity image after the geometric transformation to the display 114.

Then, the processing of the SWS sequence illustrated in FIG. 10 finishes. The ultrasonic elastic-modulus measurement processing described above enables the calculation of the elastic-modulus frame data elf in the SWS sequence.

3. Details of Processing at Step S153

At step S153, the propagation information analyzer 110 detects the wave front from the displacement-amount frame data pt of the observation point Pij in the region of interest roi in each transmission event.

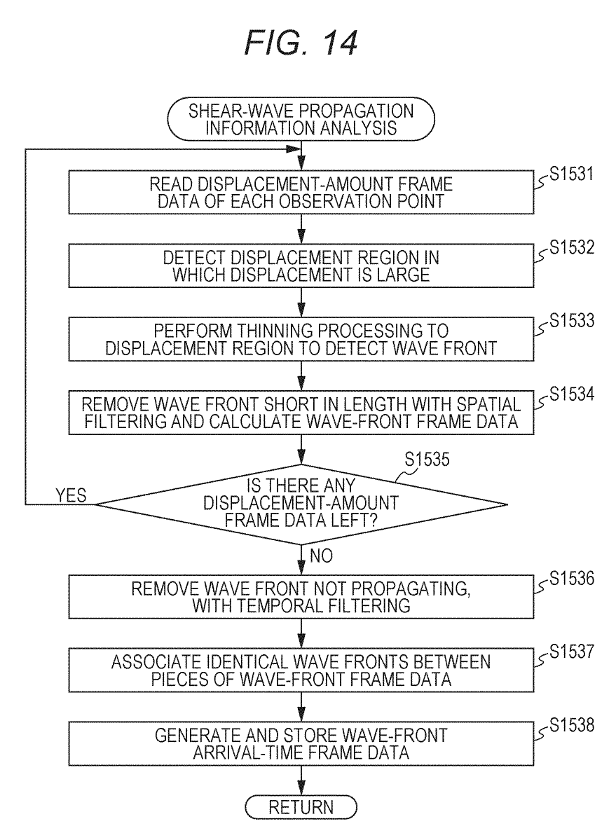

The description will be given in detail with the flowchart of FIG. 14. FIG. 14 is a flowchart of the operation of propagation information analysis of the shear wave. FIGS. 15A to 15F are schematic charts of the operation of propagation analysis of the shear wave.

First, the displacement-amount frame data ptl of each observation point Pij corresponding to the transmission event, is acquired from the data storage 115 (step S1531).

Next, a displacement region in which the displacement is relatively large, is extracted (step S1532). The propagation information analyzer 110 extracts a displacement region in which the displacement is larger than a predetermined threshold value, from the displacement-amount frame data ptl.

The description will be given below with the schematic charts of FIGS. 15A to 15F.

FIG. 15A illustrates an exemplary displacement image expressed by the displacement-amount frame data. Similarly to FIGS. 12A to 12E, “∘” in the figure indicates part of the tissue of the object to be examined corresponding to the region of interest roi, and each intersection of broken lines indicates a position before the application of the push wave pp. The x axis agrees with the array direction of the oscillators in the probe 101, and the z axis agrees with the depth direction of the object to be examined. The propagation information analyzer 110 extracts a region in which the amount of displacement δ is large, with a dynamic threshold value with the amount of displacement δ as a function of coordinate x every z coordinate. A region exceeding a threshold value, is extracted as a region in which the amount of displacement δ is large, with a dynamic threshold value with the amount of displacement δ as a function of coordinate z every x coordinate. The dynamic threshold value means that signal analysis or image analysis is performed in a target region to determine a threshold value. The threshold value is not constant, and varies depending on the width of a signal or the maximum value in the target region. FIG. 15A illustrates a graph 711 in which the amount of displacement on a straight line 710 at z=z₁ is plotted and a graph 721 in which the amount of displacement on a straight line 720 at x=x₁ is plotted. This arrangement enables, for example, extraction of a displacement region 730 in which the amount of displacement δ is larger than the threshold value.

Array B of FIG. 13 indicates the displacement-amount frame data ptl calculated to each transmission event. The hatched region in each piece of displacement-amount frame data ptl, is a displacement region in which the amount of displacement δ is larger than the threshold value. As indicated in array B of FIG. 13, the displacement region moves in the X direction and the size of the displacement region increases as time passes.

Next, the propagation information analyzer 110 performs thinning processing to the displacement region, to extract the wave front (step S1533). Displacement regions 740 and 750 illustrated in the schematic chart of FIG. 15B are each a region extracted as the displacement region 730 at step S1532. The propagation information analyzer 110 extracts the wave front with, for example, a Hilditch thinning algorithm. For example, in the schematic chart of FIG. 15B, a wave front 741 and a wave front 751 are extracted from the displacement region 740 and the displacement region 750, respectively. Note that the thinning algorithm is not limited to the Hilditch, and thus any thinning algorithm may be used. For each displacement region, processing of removing coordinates at which the amount of displacement δ is not more than the threshold value, from the displacement region may be repeatedly performed with the threshold value increasing, until the displacement region is rendered into a line having a width of one pixel. The propagation information analyzer 110 outputs the extracted wave front as the wave-front frame data wfl to the data storage 115.

Next, the propagation information analyzer 110 performs spatial filtering to the wave-front frame data wfl, to remove a wave front short in length (step S1534). For example, the propagation information analyzer 110 detects the length of each wave front extracted at step S1533, and removes a wave front shorter in length than half of the average value of the lengths of all the wave fronts, as noise. Specifically, as illustrated in the wave-front image of FIG. 15C, the average value of the lengths of wave fronts 761 to 764 is calculated, and the wave fronts 763 and 764 shorter than the average value are removed as noise. This arrangement enables removal of a wave front detected wrong.

The propagation information analyzer 110 performs the operation at steps S1531 to S1534 to all the displacement-amount frame data ptl (step S1535). This arrangement allows one-by-one generation of the wave-front frame data wfl to the displacement-amount frame data ptl.

Array C of FIG. 13 indicates the wave-front frame data wfl calculated to each transmission event. An arc-shaped fine line in each piece of wave-front frame data wfl is the wave front. As indicated in array C of FIG. 13, the wave front wf moves in the X direction and the arc length of the wave front wf increases as time passes.

Next, the propagation information analyzer 110 performs temporal filtering to the plurality of pieces of wave-front frame data wfl, to remove a wave front not propagating (step S1536). Specifically, the propagation information analyzer 110 detects a temporal variation in wave-front position between at least two pieces of wave-front frame data wfl temporally in succession, to remove a wave front having an abnormal speed as noise.

The propagation information analyzer 110 detects a temporal variation in wave-front position, for example, between a wave-front image 770 at time t=t₁, a wave-front image 780 at time t=t₁+Δt, and a wave-front image 790 at time t=t₁+2Δt. For example, for a wave front 771, the propagation information analyzer 110 performs correlation processing with the wave front 771 in a region 776 in which the shear wave moves for Δt in a direction perpendicular to the wave front (x-axis direction in FIGS. 15A to 15F), around the same position as the wave front 771 in the wave-front image 780. At this time, the correlation processing is performed in a range including both of the positive direction (right side in the figure) and the negative direction (left side in the figure) of the x axis of the wave front 771. This is because a transmitted wave and a reflected wave both are detected. This arrangement detects that the destination of the wave front 771 is a wave front 781 in the wave-front image 780, to calculate the movement distance of the wave from 771 for the time Δt. Similarly, for wave front 772 or 773, correlation processing is performed in a region in which the shear wave moves for Δt in a direction perpendicular to the wave front, around the same position as the wave front 772 or 773 in the wave-front image 780. This arrangement detects that the wave front 772 has moved to the position of a wave front 783 and the wave front 773 has moved to the position of a wave front 782.

Similar processing is performed between the wave-front image 780 and the wave-front image 790, to detect that the wave front 781 has moved to the position of a wave front 791, the wave front 782 has moved to the position of a wave front 792, and the wave front 783 has moved to the position of a wave front 793. Here, one wave front indicated with each of the wave fronts 773, 782, and 792 has a movement distance considerably smaller than those of the other wave fronts (considerably slow propagation speed). Because there is a high possibility that such wave fronts are detected wrong, the wave fronts are deleted as noise. This arrangement enables detection of wave fronts 801 and 802 as illustrated in the wave-front frame data 800 of FIG. 15E.

The operations enable generation of the sequence of wave-front frame data wfl at each time. The propagation information analyzer 110 outputs the generated sequence of the plurality of pieces of wave-front frame data wfl, to the data storage 115. At this time, corresponding information to the plurality of wave fronts that is generated, may be output to the data storage 115 (step S1537). The corresponding information to the wave fronts indicates to which wave front the same wave front corresponds in each wave-front image, and means, for example, in a case where it is detected that the wave front 772 has moved to the position of the wave front 783, that the wave front 783 and the wave front 772 are the same wave front.

Next, the propagation information analyzer 110 generates the wave-front arrival-time frame data at (step S1538). Specifically, the propagation information analyzer 110 detects the positional relationship with the wave front at each time, from the wave-front frame data wfl at each time and the corresponding information to the wave fronts.

The generation of the wave-front arrival-time frame data at will be described with FIG. 15R. FIG. 15F illustrates a combination of the wave-front frame data wfl at time t and the wave-front frame data wfl at time t+Δt as one piece of wave-front frame data 810. Here, there is the corresponding information indicating that a wave front 811 at the time t and a wave front 812 at the time t+Δt are the same wave front. The propagation information analyzer 110 detects, from the corresponding information, the coordinates (xₜ₊Δₜ, zₜ₊Δₜ) on the wave front 812 corresponding to the coordinates (xₜ, zₜ) on the wave front 811. This arrangement enables estimation that the shear wave passing through the coordinates (xₜ, zₜ) at the time t arrives at the coordinates (xₜ₊Δₜ, zₜ₊Δₜ) at the time t+Δt. Thus, the time t at which the wave front 811 arrives at the coordinates (xₜ, zₜ) can be associated with the time t+Δt at which the wave front 812 that is the same as the wave front 811 arrives at the coordinates (xₜ₊Δₜ, zₜ₊Δₜ). Similarly, two-dimensional interpolation calculation enables calculation of the time at which the wave front arrives at aritrary coordinates (x, z) from the wave-front position detected from the wave-front frame data wfl having determined acquisition time.

Array D of FIG. 13 illustrates the wave-front arrival-time frame data at in which one-frame collection of the wave front wf in the wave-front frame data wfl calculated to each transmission event, is plotted with the acquisition time of the wave-front frame data wfl as a function value. Each arc-shaped fine line represents the arrival time of the wave front in the wave-front arrival-tune frame data at.

4. Details of Processing at Step S155

At step S155, for the observation point Pij in the region of interest roi, the elastic-modulus calculator 111 calculates the propagation speed of the shear wave or the elastic modulus, on the basis of the wave-front arrival-time frame data ato (o is a natural number expressing the number of different wave fronts, but the wave-front arrival-time frame data at is provided in a case where the numbers are not distinguished), to calculate the elastic-modulus frame data elf to the region of interest roi.

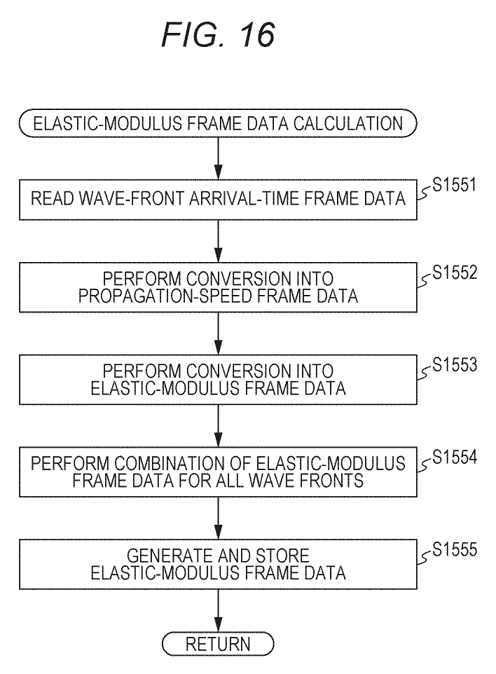

FIG. 16 is a flowchart of the operation of elastic-modulus calculation in the ultrasonic diagnostic apparatus 100. First, the propagation-speed converter 1103 reads the wave-front arrival-time frame data ato from the data storage 115 (step S1551), and converts the wave-front arrival-time frame data ato into propagation-speed frame data vfo with the following method (step S1552).

In FIG. 15F, when the distance between the coordinates (xₜ, zₜ) and the coordinates (xₜ₊Δₜ, zₜ₊Δₜ) is defined as in, the speed v(xₜ, zₜ) of the shear wave that passes through the coordinates (xₜ, zₜ), can be estimated as a value having the distance m divided by the required time Δt. That is the following expressing is satisfied: v(xₜ, zₜ)=m/Δt=√{(xₜ₊Δₜ−xₜ)²+(zₜ₊Δₜ−zₜ)}/Δt. The propagation-speed converter 1103 extracts the wave-front arrival-time frame data cat from the wave-front arrival-time frame data ato to all the wave fronts, performs the processing described above, and acquires the speed v of the shear wave for all the coordinates through which the wave front has passed.

Array E of FIG. 13 indicates the propagation-speed frame data vf in which the wave-front arrival-time frame data at calculated to each transmission event, is differentiated.

Next, the elastic-modulus calculator 111 converts the propagation-speed frame data vfo into the elastic-modulus frame data (step S1553). The elastic-modulus frame data includes the elastic modulus at each coordinates calculated on the basis of the propagation speed of the shear wave. The elastic modulus proportional to the square of the speed of the shear wave, is calculated on the basis of the following expression: el(xₜ, zₜ)=KT×v(xₜ, zₜ)². KT represents a constant, and is approximately three in the tissue of the human body.

Array F of FIG. 13 indicates the elastic-modulus frame data elf calculated by the above expression from the propagation-speed frame data vf.

On the basis of the procedure, the elastic-modulus calculator 111 combines elastic-modulus frame data elo (o is a natural number expressing the number of different wave fronts, but the elastic-modulus frame data el is provided in a case where the numbers are not distinguished) for all the wave fronts o (step S1554). The elastic-modulus calculator 111 averages the elastic-modulus frame data elo for all the wave fronts o, with the coordinates ij as an indicator, generates one frame of elastic-modulus frame data elf, and stores the one frame of elastic-modulus frame data elf into the data storage 115 (step S1555).

Then, the elastic-modulus-measurement calculation processing based on the propagation analysis of the shear wave, finishes.

5. Details of Processing at Step S150

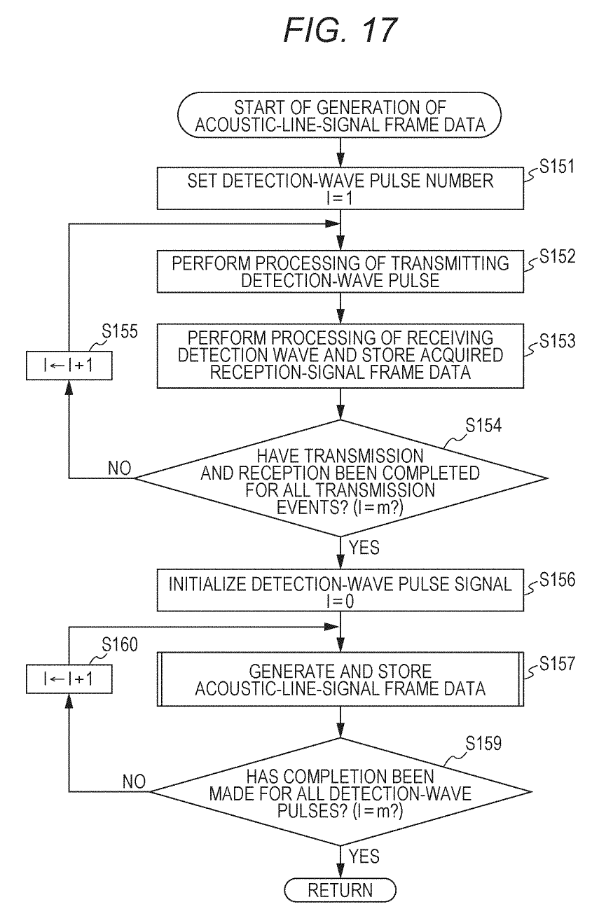

The overview of generation processing of the acoustic-line-signal frame data dsl at step S150, will be described. FIG. 17 is a flowchart of the operation of beam forming of the detection-wave receiver 108.

First, the identification number 1 of the detection wave is set 1 (step S151). The transmitter 106 performs the transmission processing (transmission event) of transmitting the detection-wave pulse pwpl for transmission of the detection wave pwl, to each oscillator included in the detection-wave transmission oscillator array Tx in the plurality of oscillators 101a included in the probe 101 (step S152).

Next, the detection-wave receiver 108 generates the reception signal rfk on the basis of the electric signal from the reflected wave at the probe 101, outputs the reception signal rfk to the data storage 115, and stores the reception signal rfk into the data storage 115 (step S153). It is determined whether the transmission and reception of the detection wave have been completed for all the number m of prescribed transmission events (step S154). In a case where the completion has not been made, 1 is incremented (step S155) and the processing goes back to step S152. Then, the transmission event is performed from the detection-wave transmission oscillator array Tx. In a case where the completion has been made, the processing proceeds to step S156.

Next, the identification number 1 of the detection wave is initialized to 0 (step S156). On the basis of the reception signal rfk stored in the data storage 115, the detection-wave receiver 108 generates the acoustic line signal to the plurality of observation points Pij in the detection-wave irradiation region Ax, generates the acoustic-line-signal frame data dsl, outputs the acoustic-line-signal frame data dsl to the data storage 115, and stores the acoustic-line-signal frame data dsl into data storage 115 (step S157). The details of a method of generating the acoustic-line-signal frame data dsl at step S157, will be described later.

It is determined whether the generation of the acoustic-line-signal frame data dsl based on the detection-wave pulse pwpl has been completed for all the number m of transmission events (step S159). In a case where the completion has not been made, 1 is incremented (step S160) and the processing goes back to step S157. In a case where the completion has been made, the processing finishes.

Then, the processing at step S150 in FIG. 11 finishes.

7. Details of Processing at Step S157

The details of generation processing of the acoustic-line-signal frame data dsl at step S157, will be described.

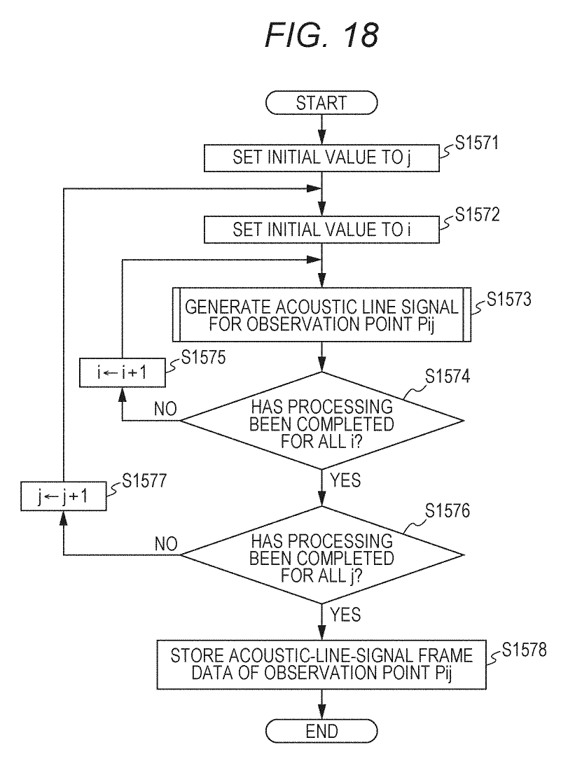

FIG. 18 is a flowchart of the generation operation of the acoustic-line-signal frame data in the detection-wave receiver 108.

First, j and i are each initialized to a minimum value in the detection-wave irradiation region Ax (steps S1571 and S1572). Next, the detection-wave receiver 108 generates the acoustic line signal dsij for the observation point Pij (step S1573). The details of the processing at step S1573 will be described later.

Next, it is determined whether the processing has been completed for all i in the detection-wave irradiation region Ax (step S1574). In a case where the completion has not been made, i is incremented (step S1575) and the acoustic line signal is generated for the observation point Pij (step S1573). It is determined whether the processing has been completed for all j in the detection-wave irradiation region Ax (step S1576). In a case where the completion has not been made, j is incremented (step S1577), and the acoustic line signal is generated for the observation point Pij (step S1573). In a case where the completion has been made for all i and all j, the processing proceeds to step S1578.

At this stage, the acoustic line signal dsij is generated for the observation point Pij in the detection-wave irradiation region Ax in the transmission event for one time, and the acoustic-line-signal frame data dsl is generated. At step S1578, the generated acoustic-line-signal frame data dsl is output to and is stored in the data storage 115.

Then, the processing at step S157 in FIG. 17, finishes.

8. Details of Processing at Step S1573

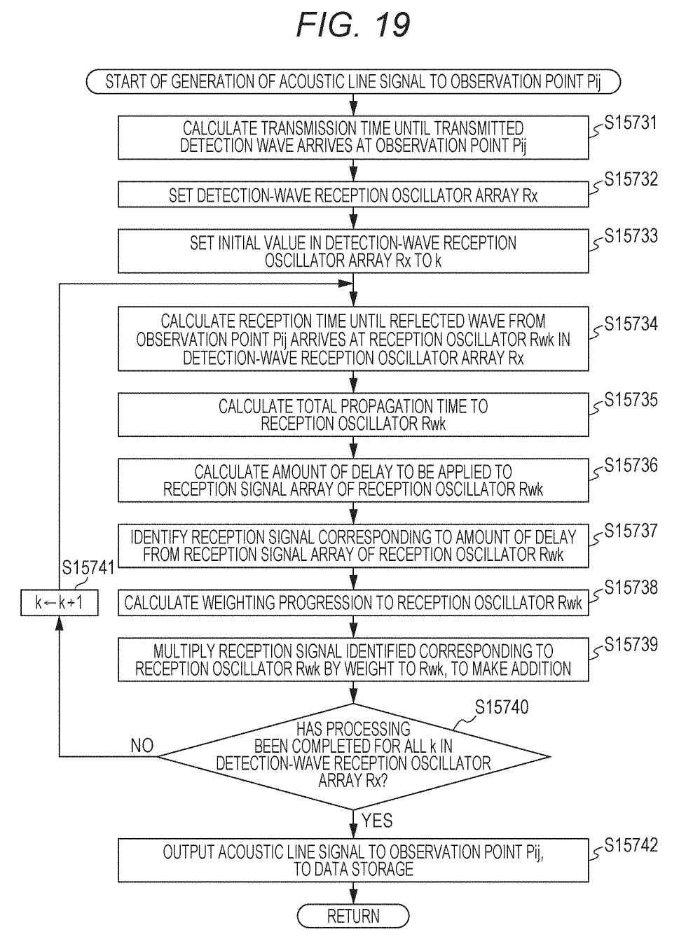

Next, the operation of generation processing of the acoustic line signal for the observation point Pij at step S1573, will be described. FIG. 19 is a flowchart of the generation operation of the acoustic line signal for the observation point Pij in the detection-wave receiver 108.

First, at step S15731, for an arbitrary observation point Pij included in the detection-wave irradiation region Ax, the delay processor 10831 calculates the transmission time until the transmitted ultrasonic wave arrives at the observation point Pij in the object to be examined. As described above, the transmission path to the observation point Pij is calculated as the shortest path 401 in which the detection wave pwl generated perpendicularly to the oscillator array from the detection-wave transmission oscillator array Tx, arrives at the observation point Pij. The length of the transmission length is divided by the speed of sound cs of the ultrasonic wave, so that the transmission time can be calculated.

Next, the detection-wave pulse reception oscillator array Rx is set (step S15732).

Next, the oscillator identification number k of the reception oscillator Rwk in the detection-wave reception oscillator array Rx is initialized to a minimum value in the detection-wave reception oscillator array Rx (step S15733). The reception time until the reflected wave arrives at the reception oscillator Rwk of the detection-wave reception oscillator array Rx after the transmitted detection wave is reflected on the observation point Pij in the object to be examined, is calculated (step S15734). The geometrically determined length of the path 402 from the observation point Pij to the reception oscillator Rwk, is divided by the speed of sound cs of the ultrasonic wave, so that the reception time can be calculated. Furthermore, from the total of the transmission time and the reception time, the total propagation time until the ultrasonic wave transmitted from the detection-wave transmission oscillator array Tx reflects on the observation point Pij and then arrives at the reception oscillator Rwk, is calculated (step S15735). On the basis of the difference in the total propagation time to each reception oscillator Rwk in the detection-wave reception oscillator array Rx, the amount of delay to each reception oscillator Rwk is calculated (step S15736).

At step S15737, the delay processor 10831 identifies, as a reception signal based on the reflected wave from the observation point Pij, the reception signal rfk corresponding to the time from which the amount of delay to each reception oscillator Rwk is subtracted, from the reception signal array corresponding to the reception oscillator Rwk in the detection-wave reception oscillator array Rx.

Next, a weight calculator (not illustrated) calculates the reception apodization to each reception oscillator Rwk such that the weight is maximum to the oscillator located at the center in the array direction of the detection-wave reception oscillator array Rx (step S15738). The adder 10832 multiplies the reception signal rfk identified corresponding to each reception oscillator Rwk by the weight to each reception oscillator Rwk, makes an addition, and calculates the acoustic line signal dsij to the observation point Pij (step S15739).

It is determined whether the calculation processing of the acoustic line signal dsij has been completed for all the reception oscillators Rwk included in the detection-wave reception oscillator array Rx (step S15740). In a case where the completion has not been made, k is incremented (step S15741), and furthermore the amount of delay for the reception oscillator Rwk is calculated (step S15739). In a case where the completion has been made, the processing proceeds to step S15742. At this stage, the acoustic line signal dsij to the observation point Pij is calculated for all the reception oscillators Rwk included in the detection-wave reception oscillator array Rx. The calculated acoustic line signal dsij to the observation point Pij is output to and is stored in the data storage 115 (step S15762).

Then, the processing at step S1573 in FIG. 18, finishes.

<Effect>

1. Propagation of Shear Wave Due to Push Wave Pp

The propagation characteristic of the shear wave due to the push wave pp, will be described.

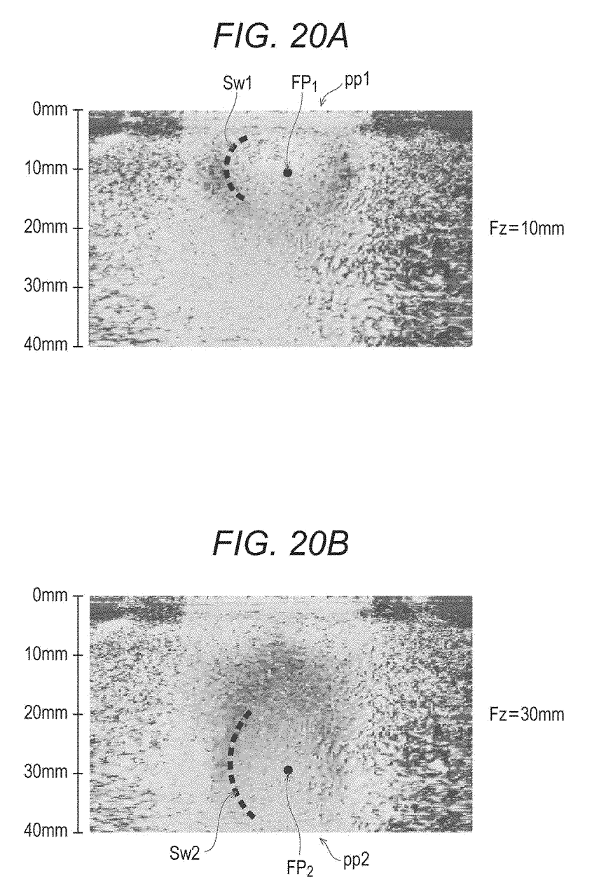

FIGS. 20A and 20B each illustrate a simulated result of an aspect of shear due to the push wave in the ultrasonic diagnostic apparatus 100. FIG. 20A illustrates an aspect in which the wave front Sw1 of the shear wave propagates in a shallow portion of the object to be examined (10 mm from the body surface). FIG. 20B illustrates an aspect in which the wave front Sw2 of the shear wave propagates in a deep portion of the object to be examined (30 mm from the body surface).

As illustrated in FIG. 20A, in a case where the transmission focus PF1 of the push wave pp1 is located in the shallow portion of the object to be examined, the wave front Sw1 of the shear wave having an are shape is observed. In contrast to this, as illustrated in FIG. 20B, in a case where the transmission focus PF1 of the push wave pp2 is located in the deep portion of the object to be examined, the wave front Sw2 of the shear wave having a vertically elongate elliptical arc shape is observed.

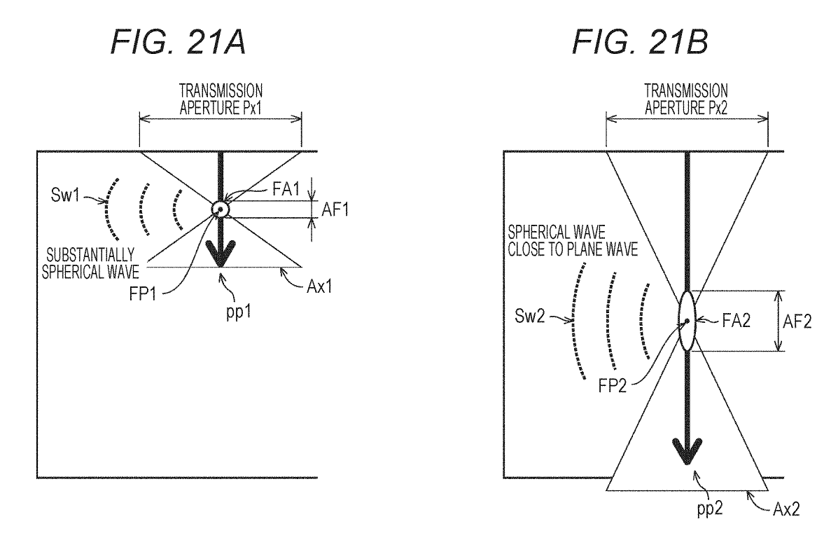

FIGS. 21A and 21B corresponding to FIGS. 20A and 20B, respectively, are schematic views of aspects in which the shear wave propagates on the basis of the push wave in the ultrasonic diagnostic apparatus 100. As illustrated in FIG. 21A, in a case where the transmission focus PP1 of the push wave ppn (n=1, 2) is located at the shallow portion of the object to be examined, the focus region FAn is formed with the region in which the energy density of the ultrasonic beam is the predetermined value or more, including the transmission focus FPn, to the transmission focus FPn of the push wave ppn. At this time, because the ultrasonic diagnostic apparatus 100 has the ratio of the depth of the transmission focus FPn to the array length of the push-wave transmission oscillator array Pxn, larger in the deeper portion of the object to be examined and smaller in the shallower portion of the object to be examined, when viewed two-dimensionally, the shape of the focus region FAn is closer to a circular shape in the shallower portion of the object to be examined and is closer to a vertically elongate elliptical shape in the deeper portion of the object to be examined. According to another scale, the length AFn in the depth direction of the focus region FAn is longer in the deeper portion of the object to be examined and is shorter in the shallower portion of the object to be examined. Therefore, as observed in FIGS. 20A and 20B, the shape of the wave front Swn of the shear wave propagating radially around the transmission focus FPn, is closer to a circular shape in the shallower portion of the object to be examined and is closer to a vertically elongate elliptical shape in the deeper portion of the object to be examined.

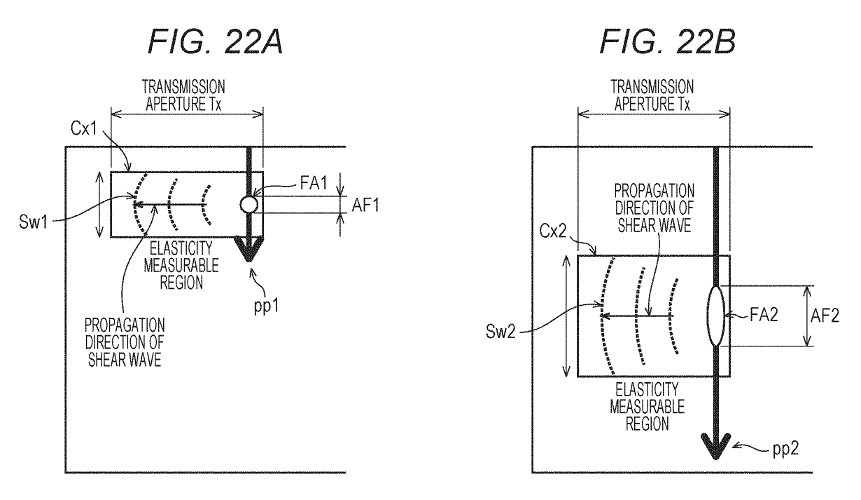

FIGS. 22A and 22B are schematic views of a tissue-displacement measurable region with the shear wave based on the push wave in the ultrasonic diagnostic apparatus 100. Because the push wave ppn (n=1, 2) oscillates, in the depth direction, the tissue of the object to be examined in the vicinity of the transmission focus FPn, the shear wave having a hypocenter at the transmission focus FPn is a transverse wave having the maximum amplitude in the depth direction of the object to be examined. The shear wave propagates radially around the transmission focus FPn, and the wave front Swn of the shear wave expands concentrically around the transmission focus FPn. In order to detect the propagation state of the shear wave highly accurately from the temporal variation in displacement of the tissue of the object to be examined, favorably, the temporal variation in displacement of the tissue of the object to be examined, is measured in the bearing direction. Therefore, it is necessary that the error in angle is a predetermined value or less between the propagation direction of the shear wave and the hearing direction.

As described above, the shape of the wave front Swn of the shear wave propagating radially around the transmission focus FPn, is closer to a circular shape in the shallower portion of the object to be examined and is closer to a vertically elongate elliptical shape in the deeper portion of the object to be examined. Therefore, as illustrated in FIGS. 22A and 22B, when an elasticity measurable region Cxn is provided such that the error in angle between the propagation direction of the shear wave and the bearing direction is the predetermined value or less, the length of the elasticity measurable region Cxn in the depth direction of the object to be examined, is smaller in the shallower portion of the object to be examined and is larger in the deeper portion of the object to be examined.

2. Effect of Ultrasonic Diagnostic Apparatus 100 According to Embodiment

Next, the effect of the ultrasonic diagnostic apparatus 100, will be described.

### (1) Configurations of Example, Modification, and Comparative Examples

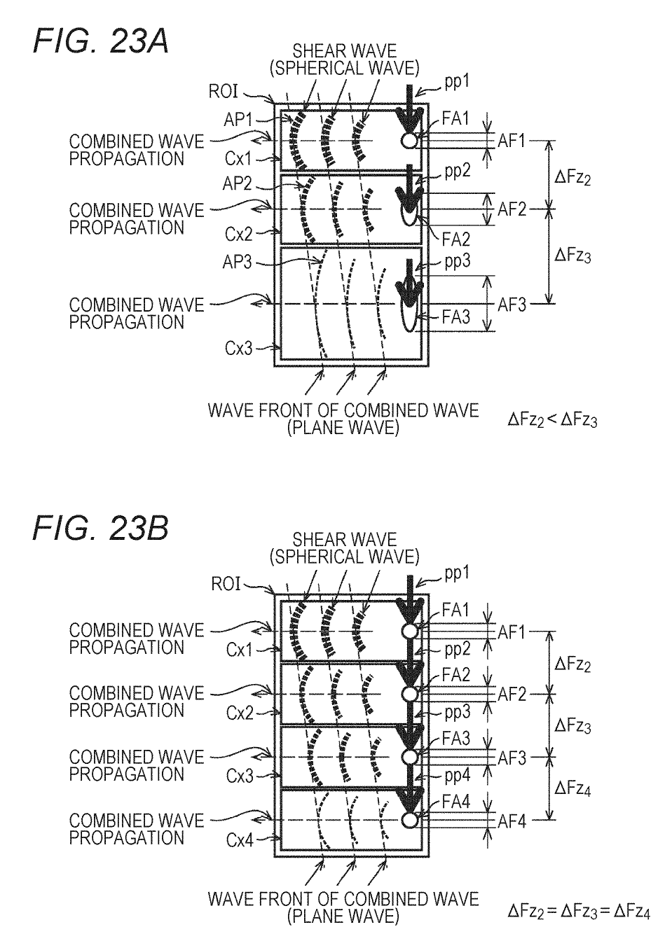

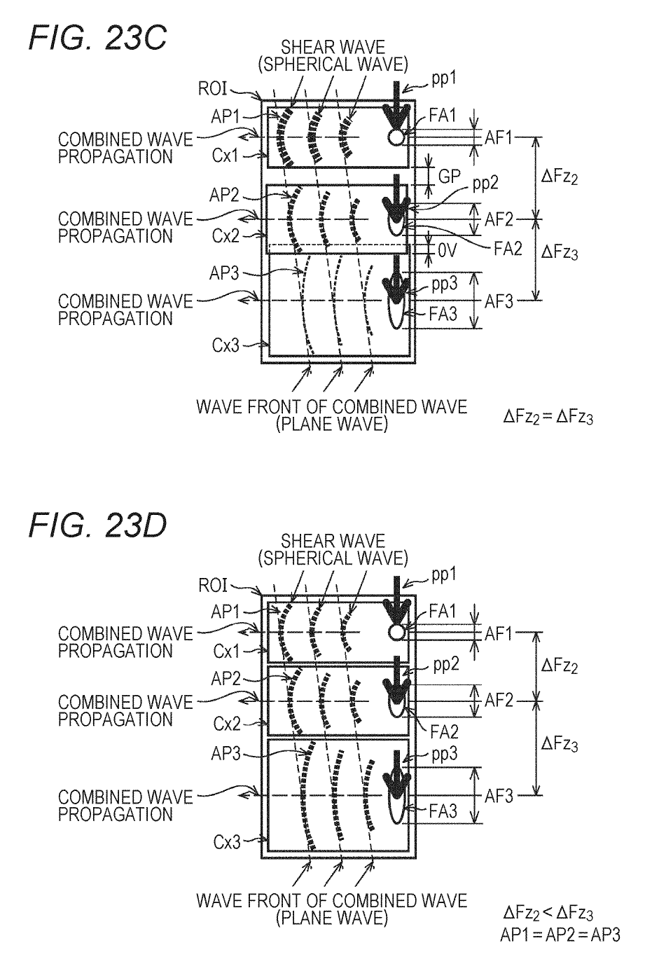

FIGS. 23A to 23D are schematic views of aspects of the measurable region with the shear wave based on the push wave. FIGS. 23A, 23B, 23C, and 23D illustrate Example of the ultrasonic diagnostic apparatus 100, Comparative Example 1, Comparative Example 2, and Modification, respectively.

Example relates to the ultrasonic diagnostic apparatus 100 according to the first embodiment, in which exemplarily with nₘₐₓ=3, the pulse width PWn every push-wave pulse pppn is constant, as illustrated in FIG. 5A, regardless of the application order for each push wave ppn (n=1, 2, 3). Note that, needless to say, the numerical value of nₘₐₓ is not limited to 3 and can be appropriately changed.

According to Modification, the pulse width PWn every push-wave pulse pppn increases in descending order every application, as illustrated in FIG. 5B, in the configuration of Example.

According to Comparative Example 1, with nₘₐₓ=4, the intervals Δfz2, Δfz3, and Δfz4 between the adjacent transmission focuses ft are equivalent. At the transmission focuses fz1 to fr4, the ratios of the depths of the transmission focuses FP1 to FP4 to the array lengths of the push-wave transmission oscillator arrays Px1 to Px4 are set equivalently, and the lengths AF1 to AF4 in the depth direction of the focus regions FA1 to FA4 are equivalent.

According to Comparative Example 2, the intervals Δfz2 and Δfz3 between the adjacent transmission focuses fz are equivalent in the configuration of Example.

### (2) Effect of Example

According to Example of the first embodiment, an increase is made in the deeper portion of the object to be examined and a decrease is made in the shallower portion of the object to be examined, for the ratios of the depths of the transmission focuses FP1 to FP3 to the array lengths of the push-wave transmission oscillator arrays Px1 to Px3. Therefore, the length AFn in the depth direction of the focus region FAn is longer in the deeper portion of the object to be examined and is shorter in the shallower portion of the object to be examined. The length of the elasticity measurable region Cxn in the depth direction of the object to be examined is larger in the deeper portion of the object to be examined and is smaller in the shallower portion of the object to be examined. According to Example, as illustrated in FIG. 23A, because an increase is made in the deeper portion of the object to be examined and a decrease is made in the shallower portion of the object to be examined, for the intervals Δfz2 and Δfz3 between the adjacent transmission focuses fz, the elasticity measurable region Cxn can be disposed in uniform density to the interval Δfzn between the transmission focuses fz, meeting the interval Δfzn between the transmission focuses fz. This arrangement enables three elasticity measurable regions Cx1 to Cx3 to cover the entire region of interest roi with no gap or no overlap.

In contrast to this, according to Comparative Example 1, as illustrated in FIG. 23B, four elasticity measurable regions Cx1 to Cx4 are required in order to cover the entire region of interest roi. Thus, transmission of the push wave ppn (n=1 to 4) to the transmission focuses fz1 to fz4, is required, so that power consumption is larger in Comparative Example 1 than in Example.

According to Comparative Example 2, as illustrated in FIG. 23C, because the intervals Δfz2 and Δfz3 between the adjacent transmission focuses fz are equivalent, the elasticity measurable region Cxn cannot meet the interval Δfzn between the transmission focuses fz. Therefore, a gap GP occurs between the elasticity measurable regions Cx1 and Cx2 in the region of interest roi. Thus, the entire region of interest rot cannot be covered. In this case, there is a possibility that the reliability of ultrasonic elastic-modulus measurement deteriorates in a portion of the gap GP Meanwhile, an overlap OV occurs between the elasticity measurable regions Cx2 and Cx3. Therefore, the power consumption required for the transmission of the push wave ppn, is not necessarily used optimally to the requirement for performance of elasticity measurement to the entire region of interest roi, and thus the power consumption is used wastefully.

Meanwhile, according to Example, as described above, because the three elasticity measurable regions Cx1 to Cx3 can cover the entire region of interest roi with no gap or no overlap, highly reliable ultrasonic elastic-modulus measurement can be performed to the entire region of interest roi with less power consumption than that in Comparative Example 1.

### (3) Effect of Modification

According to Example, the pulse width PWn every push-wave pulse pppn in the transmission of each push wave ppn, is constant. Therefore, the acoustic radiation pressure generated from each push-wave transmission oscillator array Pxn is the same, but the push wave ppn attenuates increasingly in the deeper portion of the object to be examined. Thus, the acoustic radiation pressure generated from each push wave ppn, attenuates increasingly in the deeper portion of the object to be examined, in proximity to the transmission focus fan. Because the elasticity measurable region Cxn is larger in area in the deeper portion of the object to be examined, the average acoustic radiation pressure in the elasticity measurable region Cxn decreases increasingly in the deeper portion of the object to be examined. Furthermore, in the transmission and reception of the detection wave pw, because the detection wave pw attenuates increasingly in the deeper portion of the object to be examined, the signal S/N tends to decrease increasingly in the deeper portion of the object to be examined.

In contrast to this, according to Modification, the pulse width PWn for each push-wave pulse pppn (n=1, 2, 3) increases in descending order every application in the configuration of Example, and thus the acoustic radiation pressure generated from each push-wave transmission oscillator array Pxn increases increasingly in the deeper portion of the object to be examined. This arrangement enables compensation of the attenuation of the push wave ppn in the tissue of the object to be examined, so that the difference in acoustic radiation pressure in proximity to the transmission focus fzn due to each push wave ppn, can be reduced regardless of the depth of the object to be examined. Furthermore, the acoustic radiation pressure in proximity to the transmission focus fan due to each push wave ppn increases increasingly in the deeper portion of the object to be examined, so that the average acoustic radiation pressure in the elasticity measurable region Cxn can be made constant regardless of the depth of the object to be examined.

As a result, according to Modification, in addition to that the three elasticity measurable regions Cx1 to Cx3 cover the entire region of interest roi with no gap or no overlap, similarly to Example, the pulse width PWn every push-wave pulse pppn is controlled so as to meet the time interval of the application start time PTn every push-wave pulse pppn. Thus, the shear wave or the signal S/N of the detection wave is inhibited from decreasing in the deeper portion of the object to be examined, so that more highly reliable ultrasonic elastic-modulus measurement can be performed to the entire region of interest roi. That is, power distribution is made properly in terms of the depth of the transmission focus fzn in the object to be examined, so that more highly reliable ultrasonic elastic-modulus measurement can be performed to the entire region of interest roi with less power consumption.

### SUMMARY

In the ultrasonic diagnostic apparatus 100 according to the first embodiment, the push-wave pulse transmitter 1041 supplies, a plurality of times, the push-wave pulse pppn that is set with a predetermined phase and has a predetermined time length, to each of the plurality of transmission oscillators Px selected from the plurality of oscillators 101a, to cause the plurality of transmission oscillators Tx to sequentially transmit the plurality of push waves ppn to focus onto the plurality of transmission focuses FPn different in position in the depth direction of the object to be examined. Furthermore, the plurality of push waves is transmitted such that the interval Δfzn between the adjacent transmission focuses FPn is larger in the deeper portion of the object to be examined and is smaller in the shallower portion of the object to be examined and the ratio of the depth of the transmission focus fzn to the array length Px of the plurality of transmission oscillators is larger in the deeper portion of the object to be examined and is smaller in the shallower portion of the object to be examined.

The configuration enables the elasticity measurable region Cxn to each push wave ppn, to cover the entire region of interest roi with no gap or no overlap, so that highly reliable ultrasonic elastic-modulus measurement can be achieved to the entire region of interest roi with less power consumption.

The supply time of the push-wave pulse to be supplied from the push-wave pulse transmitter may be longer in the deeper portion of the object to be examined and may be shorter in the shallower portion of the object.

The pulse width PWn every push-wave pulse pppn is controlled so as to meet the depth of the object to be examined, with the configuration, to inhibit the shear wave or the signal S/N of the detection wave from decreasing in the deep portion of the object to be examined. Thus, more highly reliable ultrasonic elastic-modulus measurement can be performed to the entire region of interest roi. That is, power distribution is made properly so as to meet the depth of the transmission focus fzn in the object to be examined, so that more highly reliable ultrasonic elastic-modulus measurement can be performed to the entire region of interest roi with less power consumption.

### Second Embodiment

In the ultrasonic diagnostic apparatus 100 according to the first embodiment, the propagation-speed converter 1103 calculates the propagation speed of the shear wave on the basis of the wave-front arrival-time frame data ato, for the observation point Pij in the region of interest roi.

However, an ultrasonic diagnostic apparatus 100A according to a second embodiment, is different from that according to the first embodiment in that propagation-speed frame data is calculated on the basis of displacement-amount frame data ptl and the propagation-speed frame data is converted into elastic-modulus frame data.

The ultrasonic diagnostic apparatus 100A will be described below.

<Configuration>

The ultrasonic diagnostic apparatus 100A is different from that according to the first embodiment in terms of the configurations of a propagation information analyzer and an elastic-modulus calculator, and thus the configurations according to the ultrasonic diagnostic apparatus 100A, will be described. The configuration except the above is the same as that of the ultrasonic diagnostic apparatus 100, and thus the description thereof will be omitted.

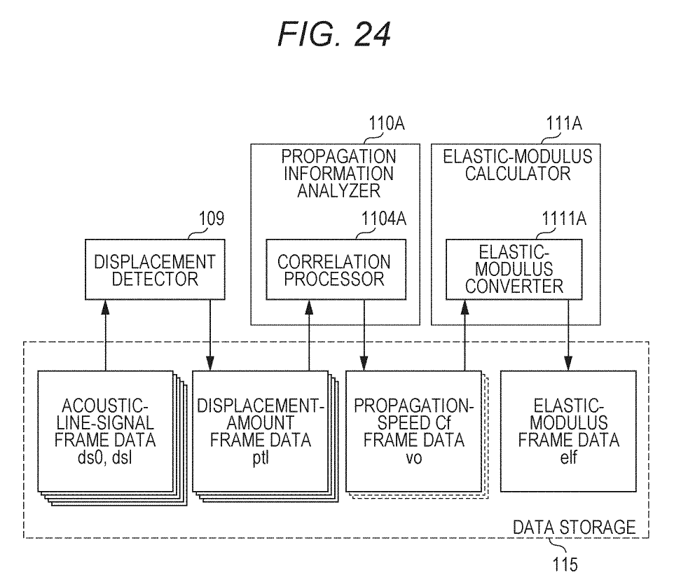

FIG. 24 is a functional block diagram of the configurations of a displacement detector 109, a propagation information analyzer 110A, and an elastic-modulus calculator 111A in the ultrasonic diagnostic apparatus 100A. The configuration of the propagation information analyzer 110A different from that in the ultrasonic diagnostic apparatus 100, will be described from the above.

The propagation information analyzer 110A is different from that in the ultrasonic diagnostic apparatus 100 in terms of including a correlation processor 1104A.

Every transmission event, the correlation processor i 104A directly generates, with cross-correlation processing, propagation-speed Cf frame data vo (o is a natural number expressing the number of different wave fronts, but propagation-speed Cf frame data v is provided in a case where the numbers are not distinguished) from a sequence of displacement-amount frame data ptl, and outputs the propagation-speed Cf frame data vo to a data storage 115.

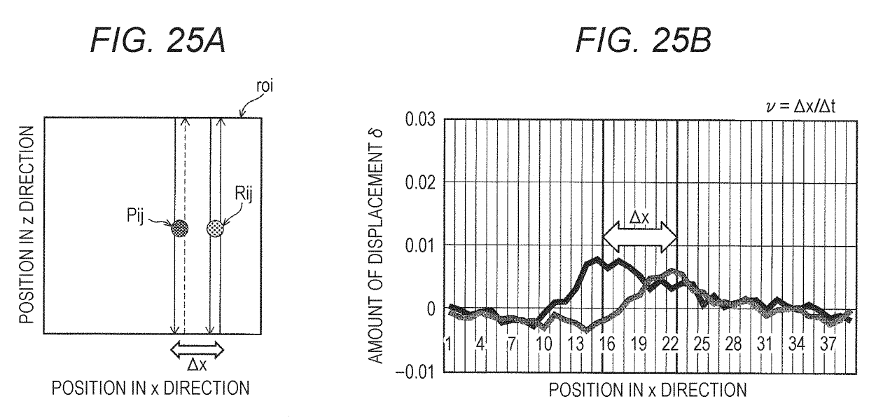

FIGS. 25A and 25B are a schematic view and a graph each illustrating the operation of propagation analysis of a shear wave in the ultrasonic diagnostic apparatus 100A. As illustrated in FIG. 25A, an observation point Pij and a reference observation point Rij that are the same in position in the z direction and are spaced apart by a predetermined distance Δx, are defined in a region of interest roi. As illustrated in FIG. 25B, the correlation processor 1104A extracts time-series variation data of displacement ptij at each of the target observation point Pij and the reference observation point Rij, on the basis of the sequence of displacement-amount frame data ptl, performs the cross-correlation processing between the plurality of pieces of time-series variation data, and calculates the transition time Δt of the displacement ptij between the target observation point Pij and the reference observation point Rij. Then, the predetermined distance Δx is divided by the transition time Δt to calculate the propagation speed vij of the shear wave to the target observation point Pij. The processing calculates the propagation speed vij of the shear wave for each target observation point Pij in the region of interest roi, so that the propagation-speed Cf frame data vo is directly generated.

With the propagation-speed Cf frame data vo as an input, an elastic-modulus converter 1111A converts propagation speed data Cfvij into elastic-modulus data elij at the observation point Pij. Thus, elastic-modulus frame data elf is generated to the region of interest roi, and the generated elastic-modulus frame data elf is output to the data storage 115 through a controller 116.

<Operation>

The operation of an SWS sequence of the ultrasonic diagnostic apparatus 100A, will be described.

The operation of the SWS sequence of the ultrasonic diagnostic apparatus 100A is different from the operation of that of the ultrasonic diagnostic apparatus 100 in terms of part in the flow of FIG. 14 illustrating the details of the operation of the propagation information analysis of the shear wave, according to step S153 in the operation flow of the SWS sequence of the ultrasonic diagnostic apparatus 100 illustrated in FIG. 11. Therefore, the different operation will be described below.

1. Operation of Propagation Information Analysis of Shear Wave

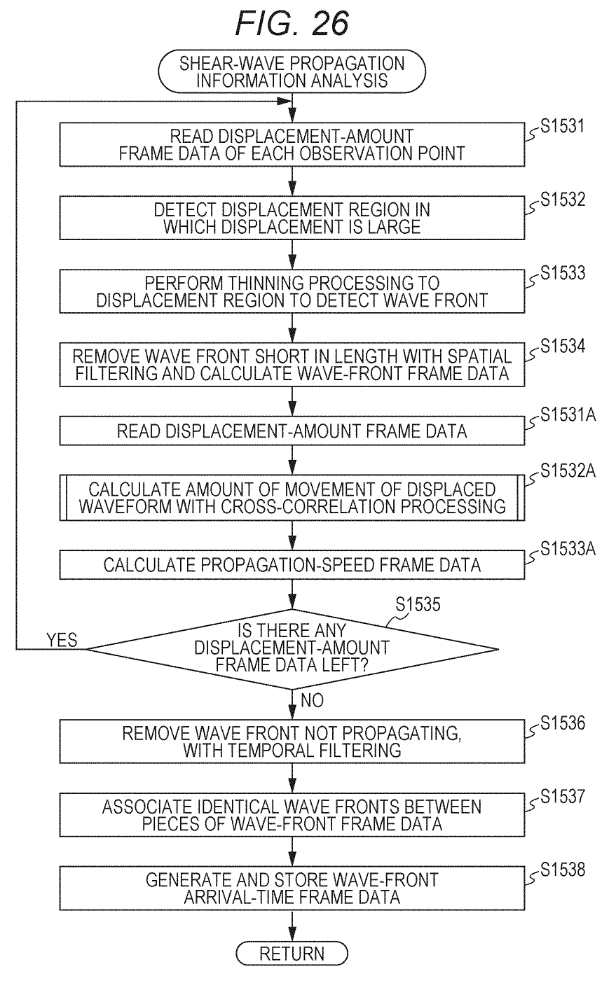

FIG. 26 is a flowchart of the operation of propagation information analysis of the shear wave in the ultrasonic diagnostic apparatus 100A. The operation from step S1531 to step S1534 and the operation from step S1535 to step S1538 are the same as those in the ultrasonic diagnostic apparatus 100 illustrated in FIG. 14, and thus the descriptions thereof will be omitted. In the ultrasonic diagnostic apparatus 100A, after step S1534, the correlation processor 1104A reads the sequence of displacement-amount frame data ptl from the data storage 115 (step S1531A), performs the cross-correlation processing between the pieces of time-series variation data of the displacement ptij at the target observation point Pij and the reference observation point Rij spaced apart by the predetermined distance Δx, calculates the transition time Δt of the displacement ptij between the target observation point Pij and the reference observation point Rij (step S1532A), and makes Δx divided by Δt to calculate the propagation speed vij of the shear wave to the target observation point Pij. The processing is performed for each target observation point Pij in the region of interest roi, to generate the propagation-speed Cf frame data vo, directly (step S1533A).

2. Description of Method of Calculating Propagation Speed Vij of Shear Wave with Cross-Correlation Processing

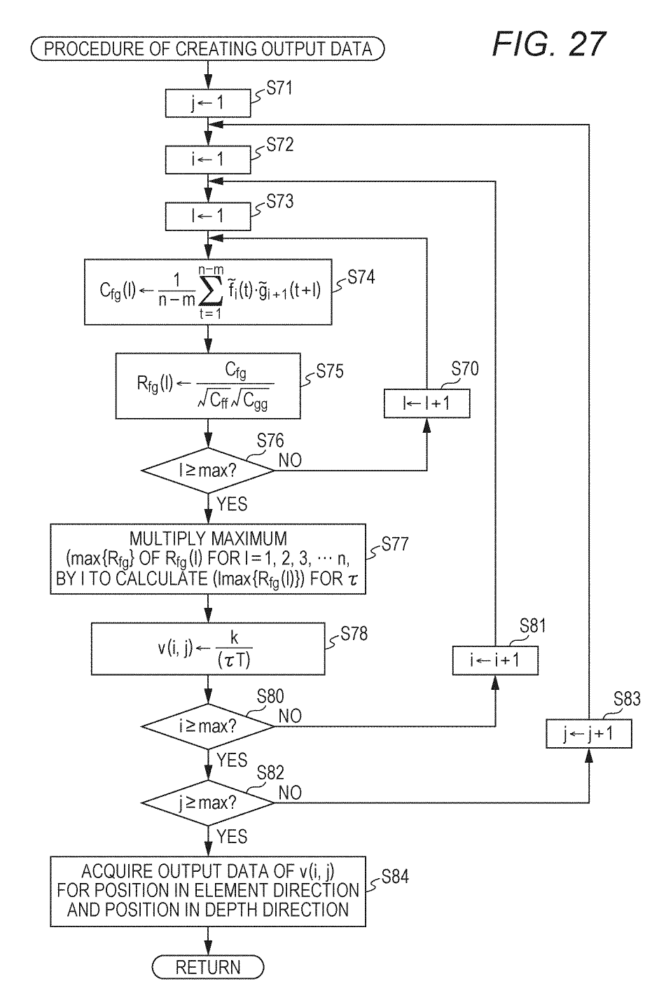

FIG. 27 is a flowchart of a procedure of creating output data of the propagation speed vij of the shear wave that can be used at steps S1532A and S1533A in FIGS. 25A and 25B.

The present flowchart includes a loop for the variable l, a loop for the variable i, and a loop for the variable j that are multiplexed. The loop for the variable l is a loop for the variable l meaning the lag between the arrival time of a wave front at position i in the x direction (oscillator array direction) and the arrival time of the wave front at position i+1 in the x direction, and is a loop for calculating Rfg and Cfg for various values that the variable l can have. Here, fi(t) represents the amount of variation in the position of the wave front in the temporal-axis direction at the position (i) in the x direction, and gi+1(t) represents the amount of variation in the position of the wave front in the temporal-axis direction at the position (i+1) in the x direction. The loop for the variable i is a loop for calculating the propagation speed of the shear wave for each of a plurality of positions in the x direction at one position in the z direction (depth direction of the object to be examined). The loop for the variable j is a loop for repeating v(i, j) for each position in the z direction.

At step S71, the variable j expressing each position in the z direction is initialized to 1. At step S72, the variable i expressing each position in the oscillator array direction is initialized to 1. At step S73, the variable l is initialized to 1. At step S74, a product-sum operation is performed to the variable l set with one value due to variable initialization or variable updating, to calculate the correlation value Cfg between fi(t) and gi+1(t+1). At step S75, the correlation value Cfg is normalized to acquire the normalized correlation value Rfg. Step S76 relates to the end requirement for the variable l. In a case where l has not reached the maximum value max, the variable l is incremented (step S70). Then, the processing goes back to step S74. Until the variable l reaches the maximum value max, the increment of the variable l and the calculation and normalization of Cfg are repeated. In a case where l has reached the maximum value max (Yes at step S76), the processing proceeds to step S77. At step S77, the maximum of Rfg for each value of l=1, 2, 3, 4 . . . n, is multiplied by the variable l to calculate the amount of lag τ in the temporal direction. Then, at step S78, a local value of the speed of the shear wave is calculated by calculation for v←k/(τT), so that the propagation speed v(i, j) of the shear wave at coordinates (i, j) is acquired.

At step S80, it is determined whether the variable i has reached the maximum value max. In a case where the variable i has not reached the maximum value max, the variable i is incremented (step S81). Then, the processing goes back to step S73. Step 582 relates to the end requirement for the loop of the variable j. In a case where the variable j has not reached the maximum value max (No at step S82), the variable j is incremented at step S83. Then, the processing goes back to step S72. When the variable j reaches the maximum value max, the processing exits from the loop. Step S84 relates to postprocessing after all the loops finish. The output data of the propagation speed v(i, j) of the shear wave in position in the x direction and the z direction, is acquired.

As described above, the present calculation method enables improvement of apparent temporal resolution and spatial resolution. This arrangement enables calculation of the speed of the shear wave that increases locally instantaneously due to a passage through hard tissue, so that elasticity evaluation can be performed highly accurately.

Then, the processing of the SWS sequence finishes. The ultrasonic elastic-modulus measurement processing of the ultrasonic diagnostic apparatus 100A described above, enables calculation of the elastic-modulus frame data elf in the SWS sequence.

<Effect>

As described above, in the ultrasonic diagnostic apparatus 100A according to the second embodiment, the propagation information analyzer 110A calculates the time-series variation data of the displacement at each of the target observation point and the reference observation point spaced apart by the predetermined distance, on the basis of the sequence of displacement-amount frame data pd, performs the cross-correlation processing between the plurality of pieces of time-series variation data, calculates the transition time of the displacement between the target observation point and the reference observation point, makes the predetermined distance divided by the transition time, calculates the propagation speed of the shear wave to the target observation point, and calculates the propagation-speed Cf frame data vo of the shear wave with the plurality of observation points in the region of interest as the target observation point.

The configuration enables direct generation of the propagation-speed Cf frame data vo from the sequence of displacement-amount frame data ptl with the cross-correlation processing in the ultrasonic elastic-modulus measurement, so that computing load can be reduced. This arrangement enables high-accurate calculation of the propagation-speed frame data and the elastic-modulus frame data with improvement in temporal resolution and spatial resolution, in addition to the effect according to the first embodiment

<Other Modifications>

Note that the present invention has been described on the basis of the embodiments, but the present invention is not limited to the embodiments. Thus, the present invention includes the following cases.

In the ultrasonic diagnostic apparatus 100 according to the embodiment, the configurations of the transmitter 106, the detection-wave receiver 108, the displacement detector 109, the propagation information analyzer 110, and the elastic-modulus calculator 111 can be appropriately changed other than the configurations described in the embodiment.

For example, although all the oscillators 101a included in the probe 101 transmit the push wave in the embodiment, the transmitter 106 may set the push-wave transmission oscillator array Px including an oscillator array corresponding to part of the plurality of oscillators 101a included in the probe 101 and may repeat ultrasonic transmission while gradually moving the transmission oscillator array in the array direction each SWS sequence. Thus, the acoustic radiation pressure of the push wave can increase.

Although the region of interest roi is set to a partial region in the detection-wave irradiation region Ax including the oscillator array (101a) including the plurality of oscillators 101a in the embodiment, the region of interest roi may be set to the entire detection-wave irradiation region Ax that is the maximum range thereof.

In the embodiment, with the region of interest roi set to a partial portion in the detection-wave irradiation region Ax, the push-wave pulse generator 104 determines the push-wave transmission oscillator array Px including all the plurality of oscillators 101a, sets the transmission focus FP of the push wave singly in the region of interest roi. The SWS sequence in which the transmission and reception of the detection wave pwl are repeated a plurality of times to the region of interest roi, is performed to calculate the elastic-modulus frame data el for the observation point located in the region of interest roi in the SWS sequence for one time. However, with the region of interest roi set to a partial portion in the detection-wave irradiation region Ax, the push wave pp may be transmitted with gradual movement of the transmission focus FP in the array direction every SWS sequence, and additionally the transmission and reception of the detection wave pwl may be repeated a plurality of times while the target observation region is being changed in the region of interest roi on the basis of the position of the transmission focus FP. Then, combination elastic-modulus frame data emp calculated for the partial portion in the region of interest roi every SWS sequence, may be combined to calculate integrated-SWS-sequence combined elastic modulus el to the entire region of interest roi.

In the embodiment, the region in which the observation point is present has a width the same as that of the oscillator array, the region being perpendicular to the reception oscillator array.

However, the embodiment is not limited to this, and thus an arbitrary region included in the ultrasonic irradiation region may be set. For example, there may be provided a plurality of belt-shaped rectangular regions having an oscillator width with a central line that is a straight line passing through the array center of the reception oscillator array, the straight line being perpendicular to the oscillator array.

The present invention may include, for example, a computer system including a microprocessor and a memory, the memory storing a computer program, the microprocessor being to operate in accordance with the computer program. For example, a computer system may include a computer program for a diagnostic method of an ultrasonic diagnostic apparatus according to an aspect of the present invention, and may operate in accordance with the program (or may instruct each connected part to operate).

The present invention includes a computer system in which the entirety or part of the ultrasonic diagnostic apparatus and the entirety or part of a beam former include a microprocessor, a recoding medium, such as a ROM or a RAM, and a hard disk unit. The RAM or the hard disk unit stores a computer program for achieving operation similar to that of each apparatus described above. The microprocessor operates in accordance with the computer program, so that each apparatus achieves the function thereof.

The entirety or part of the constituent elements included in each apparatus described above, may be included in one system large scale integration (LSI). The system LSI includes a super multifunctional LSI manufactured with integration of a plurality of constituent parts on one chip. Specifically, the system LSI is a computer system including, for example, a microprocessor, a ROM, and a RAM. These may be individually rendered in one chip, or part or the entirety thereof may be rendered in one chip. Note that LSI may be called IC, system LSI, super LSI, or ultra LSI depending on the degree of integration. The RAM stores a computer program for achieving operation similar to that of each apparatus described above. The microprocessor operates in accordance with the computer program, so that the system LSI achieves the function thereof. For example, the present invention includes a case where a beam forming method according to an aspect of the present invention is stored as a program for LSI and the LSI is inserted into a computer to execute the predetermined program (beam forming method).

Note that a technique for circuit integration is not limited to LSI, and thus the integration may be achieved with a dedicated circuit or a general-purpose processor. A field programmable gate array (FPGA) with which a program can be written after LSI manufacture or a reconfigurable processor with which connecting or setting of an LSI internal circuit cell can be reconfigured after LSI manufacture, may be used.

Furthermore, if the advancement of semiconductor technology or derived different technology allows appearance of a technique of circuit integration that replaces LSI, integration of functional blocks may be rightfully performed with the technology.

Part or the entirety of the function of the ultrasonic diagnostic apparatus according to each embodiment, may be achieved by execution of a program with a processor, such as a CPU. There may be provided a non-transitory computer readable recording medium storing a program for performing the diagnostic method of the ultrasonic diagnostic apparatus or the beam forming method. Transport of a program or a signal that has been stored in a recording medium, may allow an independent different computer system to execute the program. Needless to say, the program can be distributed through a transport medium, such as the Internet.

In each ultrasonic diagnostic apparatus according to the embodiments, the data storage that is a storage device is located inside the ultrasonic diagnostic apparatus. However, the storage device is not limited to this, and thus a semiconductor memory, a hard disk drive, an optical disc drive, or a magnetic storage device may be connected to the ultrasonic diagnostic apparatus externally.

The division of the functional blocks in the block diagrams is exemplary. Thus, a plurality of functional blocks may be achieved as one functional block, one functional block may be divided into at least two, or part of the functions may be moved to a different functional block. The functions of a plurality of functional blocks having similar functions may be processed in parallel or on a time-division basis by a single piece of hardware or software.

The order in which the steps are performed is exemplary for the specific description of the present invention, and thus the order may be changed instead of the above. Part of the steps may be performed simultaneously with a different step (in parallel).

Each ultrasonic diagnostic apparatus is connected with the probe and the display externally, but these may be integrally included in the ultrasonic diagnostic apparatus.

In each embodiment, there is provided the probe in which the plurality of piezoelectric oscillators is disposed one-dimensionally. However, the configuration of the probe is not limited to this. For example a two-dimensional arrangement oscillator in which a plurality of piezoelectric oscillators is arranged two-dimensionally, or a rocking probe that mechanically rocks a plurality of oscillators arranged one-dimensionally to acquire a three-dimensional tomographic image, may be used. Thus, the probes can be appropriately selectively used depending on measurement. For example, in a case where a probe having oscillators arranged two-dimensionally is used, the timing at which voltage is applied to each piezoelectric oscillator or the value of voltage is changed individually, so that the irradiation position or direction of an ultrasonic beam to be transmitted can be controlled.

The probe may include the function of pan of the transmitter and receiver. For example, on the basis of a control signal for generating a transmission electric signal, output front the transmitter and receiver, the transmission electric signal is generated inside the probe and then the transmission electric signal is converted into an ultrasonic wave. In addition, a received reflected wave is converted into a reception signal, so that an acoustic line signal can be generated inside the probe, on the basis of the reception signal.

The ultrasonic diagnostic apparatus according to each embodiment may be combined with at least one of the functions of the modifications thereof. Furthermore, all the numbers used above are exemplary for the specific description of the present invention, and thus the present invention is not limited to the exemplified numbers. Furthermore, the present invention includes various modifications subjected to alterations in the scope to be conceived by a person skilled in the art to the present embodiment.

### CONCLUSION

According to the present embodiment, there is provided an ultrasonic diagnostic apparatus to which a probe including a plurality of oscillators arranged linearly is connectable and that causes the probe to transmit a push wave including an ultrasonic beam to focus into an object to be examined, to detect a propagation speed of a shear wave generated by an acoustic radiation pressure of the push wave, the ultrasonic diagnostic apparatus including: a push-wave pulse transmitter that supplies, a plurality of times, a push-wave pulse that is set with a predetermined phase and has a predetermined time length, to each of a plurality of transmission oscillators selected from the plurality of oscillators, to cause the plurality of transmission oscillators to sequentially transmit a plurality of push waves to focus onto a plurality of transmission focuses different in position in a depth direction of the object to be examined; a detection-wave pulse transmitter that supplies, after the transmission of the plurality of push waves, a detection-wave pulse to part or all of the plurality of oscillators, to cause the plurality of oscillators to transmit, a plurality of times, a detection wave to pass through a region of interest expressing a range to be analyzed in the object to be examined; and a propagation information analyzer that calculates propagation-speed frame data of a shear wave in the region of interest, based on a reflected detection wave received on a time series basis by the plurality of oscillators, the reflected wave corresponding to each of the plurality of detection waves, in which the push-wave pulse transmitter causes the transmission of the plurality of push waves such that an interval between the transmission focuses adjacent to each other is larger in a deeper portion of the object to be examined and is smaller in a shallower portion of the object to be examined and a ratio of a depth of each transmission focus to an array length of the plurality of transmission oscillators is larger in the deeper portion of the object to be examined and is smaller in the shallower portion of the object to be examined.

The configuration enables the elasticity measurable region Cxn to each push wave ppn, to cover the entire region of interest roi with no gap or no overlap, so that highly reliable ultrasonic elastic-modulus measurement can be achieved to the entire region of interest roi with less power consumption. That is the transmission position of the push wave ppn can be made properly to the requirement for performance of elasticity measurement of the entire region of interest roi, so that power consumption required for the transmission of the push wave ppn can be prevented from wasting.

According to a different aspect, in any aspect above, an interval between supply start times of the push-wave pulses to be supplied from the push-wave pulse transmitter may be longer in the deeper portion of the object to be examined and may be shorter in the shallower portion of the object to be examined.

The configuration enables the transmission of the plurality of push waves such that the interval between the transmission focuses adjacent to each other is larger in the deeper portion of the object to be examined and is smaller in the shallower portion of the object to be examined.

According to a different aspect, in any aspect above, the push-wave pulse transmitter may supply, the plurality of times, the push-wave pulse to an identical array including the transmission oscillators.

The configuration enables the transmission of the plurality of push waves easily such that the ratio of the depth of each of the transmission focuses to the array length of the plurality of transmission oscillators is larger in the deeper portion of the object to be examined and is smaller in the shallower portion of the object to be examined.

According to a different aspect, in any aspect above, a supply time of the push-wave pulse to be supplied from the push-wave pulse transmitter, may be longer in the deeper portion of the object to be examined and may be shorter in the shallower portion of the object to be examined.

The pulse width PWn every push-wave pulse pppn is controlled so as to meet the depth of the object to be examined, with the configuration, to inhibit the shear wave or the signal S/N of the detection wave from decreasing in the deep portion of the object to be examined. Thus, more highly reliable ultrasonic elastic-modulus measurement can be performed to the entire region of interest roi. That is, power distribution is made properly so as to meet the depth of the transmission focus fin in the object to be examined, so that more highly reliable ultrasonic elastic-modulus measurement can be performed to the entire region of interest i with less power consumption.

According to a different aspect, in any aspect above, when, for each of the transmission focuses, a region in which the transmission focus is located and an ultrasonic beam has energy density that is a predetermined value or more, is defined as a focus region, a length in a depth direction of the focus region may be longer in the deeper portion of the object to be examined and may be shorter in the shallower portion of the object to be examined.

The plurality of push waves is transmitted such that the ratio of the depth of each of the transmission focuses to the array length of the plurality of transmission oscillators is larger in the deeper portion of the object to be examined and is smaller in the shallower portion of the object to be examined, so that the configuration can be achieved.

According to a different aspect, in any aspect above, the push-wave pulse transmitter may specify the plurality of transmission oscillators, set the phase of the push-wave pulse to be applied for each of the transmission oscillators, voltage to be applied to the push-wave pulse and a voltage application time for each of the push-wave pulses, and a minimum voltage application start time of the push-wave pulse for each of the push-wave pulses, and supply the push-wave pulse.

The configuration enables the push-wave pulse transmitter to cause the transmission of the plurality of push waves such that the interval between the transmission focuses adjacent to each other is larger in the deeper portion of the object to be examined and is smaller in the shallower portion of the object to be examined and the ratio of the depth of each of the transmission focuses to the array length of the plurality of transmission oscillators is larger in the deeper portion of the object to be examined and is smaller in the shallower portion of the object to be examined.

According to a different aspect, in any aspect above, the detection wave may include a plane wave to propagate in the object to be examined, perpendicularly to an array including the plurality of oscillators.

The configuration enables acquisition of the acoustic line signal for the entire region of interest with transmission and reception of a one-time detection wave, and enables detection of the propagation speed of the shear wave with transmission and reception of the detection wave including the plane wave for a plurality of times during a predetermined time.

According to a different aspect, in any aspect above, there may be further provided: a detection-wave receiver that generates, based on the reflected detection wave corresponding to each of the plurality of detection waves, an acoustic line signal for a plurality of observation points in the region of interest, to generate a sequence of acoustic-line-signal frame data and a displacement detector that detects tissue displacement in the region of interest at a reception time of the reflected detection wave from the sequence of acoustic-line-signal frame data, to generate a sequence of displacement-amount frame data, in which the propagation information analyzer calculates the propagation-speed frame data of the shear wave in the region of interest, based on the sequence of displacement-amount frame data.

The configuration enables generation of the sequence of displacement-amount frame data for analysis of the propagation speed of the shear wave.

According to a different aspect, in any aspect above, the propagation information analyzer may extract a wave-front position of the shear wave from the sequence of displacement-amount frame data, at the reception time, generate a sequence of wave-front frame data, associate the wave-front position included in each of a plurality of pieces of the wave-front frame data, with the reception time, generate a sequence of wave-front arrival-time frame data, and calculate the propagation-speed frame data of the shear wave in the region of interest, based on the sequence of wave-front arrival-time frame data.

The configuration enables calculation of the propagation-speed frame data of the shear wave based on the sequence of displacement-amount frame data.

According to a different aspect, in any aspect above, the propagation information analyzer may calculate time-series variation data in displacement at each of a target observation point and a reference observation point spaced apart by a predetermined distance, based on the sequence of displacement-amount frame data, perform cross-correlation processing between the plurality of pieces of time series variation data, calculate a transition time in displacement between the target observation point and the reference observation point, make the predetermined distance divided by the transition time, calculate a propagation speed of the shear wave to the target observation point, and calculate the propagation-speed frame data of the shear wave with the plurality of observation points in the region of interest as the target observation point.

The configuration enables direct generation of the propagation-speed Cf frame data vo from the sequence of displacement-amount frame data ptl with the cross-correlation processing in the ultrasonic elastic-modulus measurement, so that computing load can be reduced.

According to a different aspect, in any aspect above, there may be further provided: an elastic-modulus calculator that calculates elastic-modulus frame data in the region of interest, based on the propagation-speed frame data of the shear wave in the region of interest.

The configuration enables easy calculation of the elastic-modulus frame data of the tissue of the object to be examined, based on the sequence of displacement-amount frame data.

According to a different aspect, in any aspect above, there may be further provided: a display that displays an image, in which the elastic-modulus calculator maps the elastic-modulus frame data in the region of interest, generates an elasticity image (elastography), converts the elasticity image into a display image, and causes the display to display the display image.

The configuration enables display of an elastic-modulus distribution image of the tissue of the object to be examined, based on the sequence of displacement-amount frame data.

According to the present embodiment, there is provided a method of controlling art ultrasonic diagnostic apparatus to which a probe including a plurality of oscillators arranged linearly is connectable and that causes the probe to transmit a push wave including an ultrasonic beam to focus into an object to be examined, to detect a propagation speed of a shear wave generated by an acoustic radiation pressure of the push wave, the method including-supplying, a plurality of times, a push-wave pulse that is set with a predetermined phase and has a predetermined time length, to each of a plurality of transmission oscillators selected from the plurality of oscillators, to cause the plurality of transmission oscillators to sequentially transmit a plurality of push waves to focus onto a plurality of transmission focuses different in position in a depth direction of the object to be examined such that an interval between the transmission focuses adjacent to each other is lager in a deeper portion of the object to be examined and is smaller in a shallower portion of the object to be examined and a ratio of a depth of each transmission focus to an array length of the plurality of transmission oscillators is larger in the deeper portion of the object to be examined and is smaller in the shallower portion of the object to be examined; supplying, after the transmission of the plurality of push waves, a detection-wave pulse to part or all of the plurality of oscillators, to cause the plurality of oscillators to transmit, a plurality of times, a detection wave to pass through a region of interest expressing a range to be analyzed in the object to be examined; generating an acoustic line signal for a plurality of observation points in the region of interest, based on a reflected detection wave corresponding to each of the plurality of detection waves, to generate a sequence of acoustic-line-signal frame data; detecting tissue displacement in the region of interest, at a reception time of the reflected detection wave, from the sequence of acoustic-line-signal frame data, to generate a sequence of displacement-amount frame data; and extracting a wave-front position of a shear wave from the sequence of displacement-amount frame data, at the reception time, generating a sequence of wave-front frame data, associating the wave-front position included in each of a plurality of pieces of the wave-front frame data, with the reception time, generating a sequence of wave-front arrival-time frame data, and calculating propagation-speed frame data of the shear wave in the region of interest, based on the sequence of wave-front arrival-time frame data.

The configuration enables the elasticity measurable region Cxn to each push wave ppn, to cover the entire region of interest roi with no gap or no overlap, so that control of the ultrasonic diagnostic apparatus capable of performing highly reliable ultrasonic elastic-modulus measurement to the entire region of interest with less power consumption, can be achieved.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasonic diagnostic apparatus to which a probe including a plurality of oscillators arranged linearly is connectable and that causes the probe to transmit a push wave including an ultrasonic beam to focus into an object to be examined, to detect a propagation speed of a shear wave generated by an acoustic radiation pressure of the push wave, the ultrasonic diagnostic apparatus comprising:
a push-wave pulse transmitter that supplies, a plurality of times, a push-wave pulse that is set with a predetermined phase and has a predetermined time length, to each of a plurality of transmission oscillators selected from the plurality of oscillators, to cause the plurality of transmission oscillators to sequentially transmit a plurality of push waves to focus onto a plurality of transmission focuses different in position in a depth direction of the object to be examined;
a detection-wave pulse transmitter that supplies, after the transmission of the plurality of push waves, a detection-wave pulse to part or all of the plurality of oscillators, to cause the plurality of oscillators to transmit, a plurality of times, a detection wave to pass through a region of interest expressing a range to be analyzed in the object to be examined; and
a propagation information analyzer that calculates propagation-speed frame data of a shear wave in the region of interest, based on a reflected detection wave received on a time series basis by the plurality of oscillators, the reflected wave corresponding to each of the plurality of detection waves,
wherein the push-wave pulse transmitter causes the transmission of the plurality of push waves such that an interval between the transmission focuses adjacent to each other is larger in a deeper portion of the object to be examined and is smaller in a shallower portion of the object to be examined and a ratio of a depth of each transmission focus to an array length of the plurality of transmission oscillators is larger in the deeper portion of the object to be examined and is smaller in the shallower portion of the object to be examined.

2. The ultrasonic diagnostic apparatus according to claim 1,
wherein an interval between supply start times of the push-wave pulses to be supplied from the push-wave pulse transmitter is longer in the deeper portion of the object to be examined and is shorter in the shallower portion of the object to be examined.

3. The ultrasonic diagnostic apparatus according to claim 1,
wherein the push-wave pulse transmitter supplies, the plurality of times, the push-wave pulse to an identical array including the transmission oscillators.

4. The ultrasonic diagnostic apparatus according to claim 1,
wherein a supply time of the push-wave pulse to be supplied from the push-wave pulse transmitter, is longer in the deeper portion of the object to be examined and is shorter in the shallower portion of the object to be examined.

5. The ultrasonic diagnostic apparatus according to claim 1,
wherein when, for each of the transmission focuses, a region in which the transmission focus is located and an ultrasonic beam has energy density that is a predetermined value or more, is defined as a focus region,
a length in a depth direction of the focus region is longer in the deeper portion of the object to be examined and is shorter in the shallower portion of the object to be examined.

6. The ultrasonic diagnostic apparatus according to claim 1,
wherein the push-wave pulse transmitter specifies the plurality of transmission oscillators, sets the phase of the push-wave pulse to be applied for each of the transmission oscillators, voltage to be applied to the push-wave pulse and a voltage application time for each of the push-wave pulses, and a minimum voltage application start time of the push-wave pulse for each of the push-wave pulses, and supplies the push-wave pulse.

7. The ultrasonic diagnostic apparatus according to claim 1,
wherein the detection wave includes a plane wave to propagate in the object to be examined, perpendicularly to an array including the plurality of oscillators.

8. The ultrasonic diagnostic apparatus according to claim 1, further comprising:
a detection-wave receiver that generates, based on the reflected detection wave corresponding to each of the plurality of detection waves, an acoustic line signal for a plurality of observation points in the region of interest, to generate a sequence of acoustic-line-signal frame data; and
a displacement detector that detects tissue displacement in the region of interest at a reception time of the reflected detection wave from the sequence of acoustic-line-signal frame data, to generate a sequence of displacement-amount frame data,
wherein the propagation information analyzer calculates the propagation-speed frame data of the shear wave in the region of interest, based on the sequence of displacement-amount frame data.

9. The ultrasonic diagnostic apparatus according to claim 8,
wherein the propagation information analyzer extracts a wave-front position of the shear wave from the sequence of displacement-amount frame data, at the reception fine, generates a sequence of wave-front frame data, associates the wave-front position included in each of a plurality of pieces of the wave-front frame data, with the reception time, generates a sequence of wave-front arrival-time frame data, and calculates the propagation-speed frame data of the shear wave in the region of interest, based on the sequence of wave-front arrival-time frame data.

10. The ultrasonic diagnostic apparatus according to claim 8,
wherein the propagation information analyzer calculates time-series variation data in displacement at each of a target observation point and a reference observation point spaced apart by a predetermined distance, based on the sequence of displacement-amount frame data, performs cross-correlation processing between the plurality of pieces of time-series variation data, calculates a transition time in displacement between the target observation point and the reference observation point, makes the predetermined distance divided by the transition time, calculates a propagation speed of the shear wave to the target observation point, and calculates the propagation-speed frame data of the shear wave with the plurality of observation points in the region of interest as the target observation point.

11. The ultrasonic diagnostic apparatus according to claim 1, further comprising
an elastic-modulus calculator that calculates elastic-modulus frame data in the region of interest, based on the propagation-speed frame data of the shear wave in the region of interest.

12. The ultrasonic diagnostic apparatus according to claim 11, further comprising
a display that displays an image,
wherein the elastic-modulus calculator maps the elastic-modulus frame data in the region of interest, generates an elasticity image, converts the elasticity image into a display image, and causes the display to display the display image.

13. A method of controlling an ultrasonic diagnostic apparatus to which a probe including a plurality of oscillators arranged linearly is connectable and that causes the probe to transmit a push wave including an ultrasonic beam to focus into an object to be examined, to detect a propagation speed of a shear wave generated by an acoustic radiation pressure of the push wave, the method comprising:
supplying, a plurality of times, a push-wave pulse that is set with a predetermined phase and has a predetermined time length, to each of a plurality of transmission oscillators selected from the plurality of oscillators, to cause the plurality of transmission oscillators to sequentially transmit a plurality of push waves to focus onto a plurality of transmission focuses different in position in a depth direction of the object to be examined such that an interval between the transmission focuses adjacent to each other is larger in a deeper portion of the object to be examined and is smaller in a shallower portion of the object to be examined and a ratio of a depth of each transmission focus to an array length of the plurality of transmission oscillators is larger in the deeper portion of the object to be examined and is smaller in the shallower portion of the object to be examined;
supplying, after the transmission of the plurality of push waves, a detection-wave pulse to part or all of the plurality of oscillators, to cause the plurality of oscillators to transmit, a plurality of times, a detection wave to pass through a region of interest expressing a range to be analyzed in the object to be examined;
generating an acoustic line signal for a plurality of observation points in the region of interest, based on a reflected detection wave corresponding to each of the plurality of detection waves, to generate a sequence of acoustic-line-signal frame data;
detecting tissue displacement in the region of interest, at a reception time of the reflected detection wave, from the sequence of acoustic-line-signal frame data, to generate a sequence of displacement-amount frame data; and
extracting a wave-front position of a shear wave from the sequence of displacement-amount frame data, at the reception time, generating a sequence of wave-front frame data, associating the wave-front position included in each of a plurality of pieces of the wave-front frame data, with the reception time, generating a sequence of wave-front arrival-time frame data, and calculating propagation-speed frame data of the shear wave in the region of interest, based on the sequence of wave-front arrival-time frame data.
